(19) United States
(12) Patent Application Publication
(10) Pub. No.: US 2002/0132320 A1
(43) Pub. Date: Sep. 19, 2002

(54) Glycoconjugate synthesis using a pathway-engineered organism

(76) Inventors: Peng George Wang, Troy, MI (US); Xi Chen, Norristown, PA (US); Ziye Liu, Detroit, MI (US); Wei Zhang, Detroit, MI (US)

Correspondence Address: BRINKS HOFER GILSON & LIONE, P.O. BOX 10395, CHICAGO, IL, 60610, US

(21) Appl. No.: 09/758,525

(22) Filed: Jan. 10, 2001

(51) Int. Cl. C12P 19/04; C12N 9/10; C12N 9/24; C12N 15/00

(52) U.S. Cl. 435/193; 435/200; 435/320.1; 435/325; 435/101

(57) ABSTRACT

This invention relates to methods and compositions for the production of glycoconjugates. In particular, organisms are provided with at least one heterologous gene encoding an enzyme for regenerating a sugar nucleotide along with at least one glycosyltransterase. Such organisms are useful for the large-scale synthesis of glycoconjugates.

### FEDERALLY SPONSORED RESEARCH OR DEVELOPMENT

The U.S. Government may have rights in the present invention pursuant to the terms of grant number A1 44040 awarded by the National Institutes of Health.

### BACKGROUND OF THE INVENTION

Advances in biological science have demonstrated that carbohydrates serve not only as energy sources or structural components, but also as key elements in a variety of molecular recognition, communication, and signal transduction events. Functions include attachment points for antibodies (e.g, human blood type A and B antigens and α-Gal oligosaccharides), receptor sites for bacterial and viral infections, cell adhesion sites for inflammation (e.g., sialyl-Lewis X antigen), and involvement in metastasis. Additionally, carbohydrates play a role in cell differentiation, development, regulation (e.g., gangliosides), protein folding (e.g., N-linked and O-linked glycan), and non-immunological defense (e.g., human milk oligosaccharide).

Despite the important biological functions and increasing demand for glycoconjugates, both chemical and enzymatic syntheses of glycoconjugates have been difficult. Large-scale production of oligosaccharides by chemical methods requires tedious protection and deprotection steps. Using chemical methods, oligosaccharides longer than a trisaccharide are not economically feasible. Enzymatic synthesis of oligosaccharides using glycosidase-catalyzed transglycosylation reactions suffers from low yields and unpredictable regio-selectivity.

Glycosyltransferases from the Leloir pathway, which are highly specific in the formation of glycosides, have proven to be a viable strategic choice for the preparative synthesis of oligosaccharides. Although a vast number of glycosyltransferases have been cloned from eukaryotic and bacterial sources, the limited access to recombinant glycosyltransferases and the prohibitive cost of the sugar-nucleotide donors prevent their application in large-scale synthesis. Thus, there remains a need for large-scale, industrial production of glycoconjugates.

In 1998, Kyowa Hakko Inc. in Japan made a significant breakthrough in large-scale synthesis of carbohydrates (Koizumi, S. et al., Nature Biotech. 1998, 16, 847-850). The key in Kyowa Hakko's technology for the large-scale production of UDP-galactose and Galα1,4Lac globotriose was a C. ammoniagenes bacterial strain engineered to efficiently convert inexpensive orotic acid to UTP. When combined with an E. coli strain engineered to over-express UDP-galactose biosynthetic genes including galK (galactokinase), galT (galactose-1-phosphate uridyltransferase), galU (glucose-1-phosphate uridyltransferase), and ppa (pyrophosphatase), UDP-galactose accumulated in the reaction solution. By combining these two strains with another recombinant E. coli strain over-expressing α1,4-galactosyltransferase gene of Neisseria gonorrhoeae, a high concentration of globotriose was obtained.

The same UDP-Galactose production system was also successfully applied in the large-scale production of disaccharide LacNAc (Endo, T. et al., Carbohydr. Res. 1999, 316, 179-183). UDP-N-acetylglucosmaine (UDP-GlcNAc) and CMP-sialic acid have been produced through a similar methodology (Tabata, K. et al., Biotech, Lett. 2000, 22, 479-483; Endo, T. et al., Appl. Microbiol. Biotechnol. 2000, 53, 257-261). The Kyowa Hakko technology is also described in EP 0861902 and EP 0870841.

Despite the significant breakthrough of Kyowa Hakko, drawbacks remain. The Kyowa Hakko processes require: (1) several plasmids in several bacterial strains; (2) transportation of intermediates in and out of the bacterial membrane to be utilized by the next enzyme; and (3) nucleotide derivatives. Thus, there remains a need for processes of producing oligosaccharides that require fewer manipulation steps and that are more cost-effective.

### BRIEF SUMMARY OF THE INVENTION

The present invention overcomes the deficiencies of the prior art and provides processes and compositions for the inexpensive, large-scale synthesis of glycoconjugates, including oligosaccharides. The present invention includes one or more of the following advantages: (1) multiple organisms are not required; (2) intermediates do not need to be transported in and out of the bacterial membrane to be utilized by the next enzyme; (3) an organism's internal energetics are used; and (4) the sugar nucleotide is regenerated.

Described herein are:

1.) Methods of producing glycoconjugates;

2.) Methods of producing sugar nucleotides;

3.) Organisms engineered to express sugar-nucleotide regeneration enzymes, glycosyltransferase enzymes, or both;

4.) Plasmids encoding at least one sugar-nucleotide regeneration enzyme; at least one glycosyltransferase, or at least one sugar-nucleotide regeneration enzyme and at least one glycosyltransferase;

5.) Systems for producing glycoconjugates;

6.) Systems for producing sugar nucleotides; and

7.) Kits containing an organism and/or plasmid of the present invention and a bioenergetic.

### BRIEF DESCRIPTION OF SEVERAL VIEWS OF THE DRAWINGS

FIG. 1 Metabolic biopathway for the synthesis of α-Gal. Five enzymes are involved including α1,3GalT (α1,3-galactosyltransferase, EC 2.4.1.151), GalT (galactose-1-phosphate uridylyltransferase, EC 2.7.7.10), GalU (glucose-1-phosphate uridylyltransferase, EC 2.7.7.9), and PykF (pyruvate kinase, EC 2.7.1.40). Metal cofactors required by individual enzymes are shown.

FIG. 2 Plasmid map of an α-Gal superbug harboring five genes encoding enzymes involved in the biosynthetic pathway of UDP-Gal regeneration and the production of α-Gal oligosaccharides. Introduced restriction enzyme sites: EcoR I, Sac II, Sal I, Xba I, Cla I. Abbreviation: rbs, ribosomal binding site.

FIG. 3 Biosynthetic pathway and corresponding plasmid map for using ATP as a bioenergetic.

FIG. 4 Biosynthetic pathway and corresponding plasmid map for using polyphosphate as a bioenergetic.

FIG. 5 Biosynthetic pathway and corresponding plasmid map for using pyruvate and O₂ as a bioenergetic.

FIG. 6 Biosynthetic pathway and corresponding plasmid for synthesis of glycoconjugates with UDP-Glc regeneration.

FIG. 7 Biosynthetic pathway and corresponding plasmid for synthesis of glycoconjugates with UDP-GlcNAc regeneration.

FIG. 8 Biosynthetic pathway and corresponding plasmid for synthesis of glycoconjugates with UDP-GalNAc regeneration.

FIG. 9 Biosynthetic pathway and corresponding plasmid for synthesis of glycoconjugates with UDP-GlcA regeneration.

FIG. 10 Biosynthetic pathway and corresponding plasmid for synthesis of glycoconjugates with CMP-NeuNAc regeneration.

FIG. 11 Biosynthetic pathway and corresponding plasmid for synthesis of glycoconjugates with GDP-Man regeneration.

FIG. 12 Biosynthetic pathway and corresponding plasmid for synthesis of glycoconjugates with GDP-Fuc regeneration.

FIG. 13 Plasmids for the regeneration of UDP-GlcNAc and UDP-GlcA that, when cotransfected into E. coli, are useful to produce hyaluronic acid.

FIG. 14 Exemplary sialic acid containing glycoconjugates.

FIG. 15 Biosynthetic pathway and corresponding plasmid map for synthesis of α-Gal using sucrose as a bioenergetic.

FIG. 16 Helicobacter pylori GDP-fucose-related gene cluster.

FIG. 17 Plasmid for GDP-fucose regeneration.

FIG. 18 Biosynthetic pathway and corresponding plasmid map for synthesis of glucose-terminated glycoconjugate using sucrose synthase.

FIG. 19 Biosynthetic pathway and corresponding plasmid map for synthesis of glucuronic acid-terminated glycoconjugate using sucrose synthase.

FIG. 20 Plasmids for the synthesis of hyaluronon through the regeneration of UDP-GlcNAc and UDP-GlcA using sucrose synthase.

FIG. 21 Biosynthetic pathway and corresponding plasmid for synthesis of glycoconjugates terminated with Galα1,4Gal sequence with UDP-Gal regeneration.

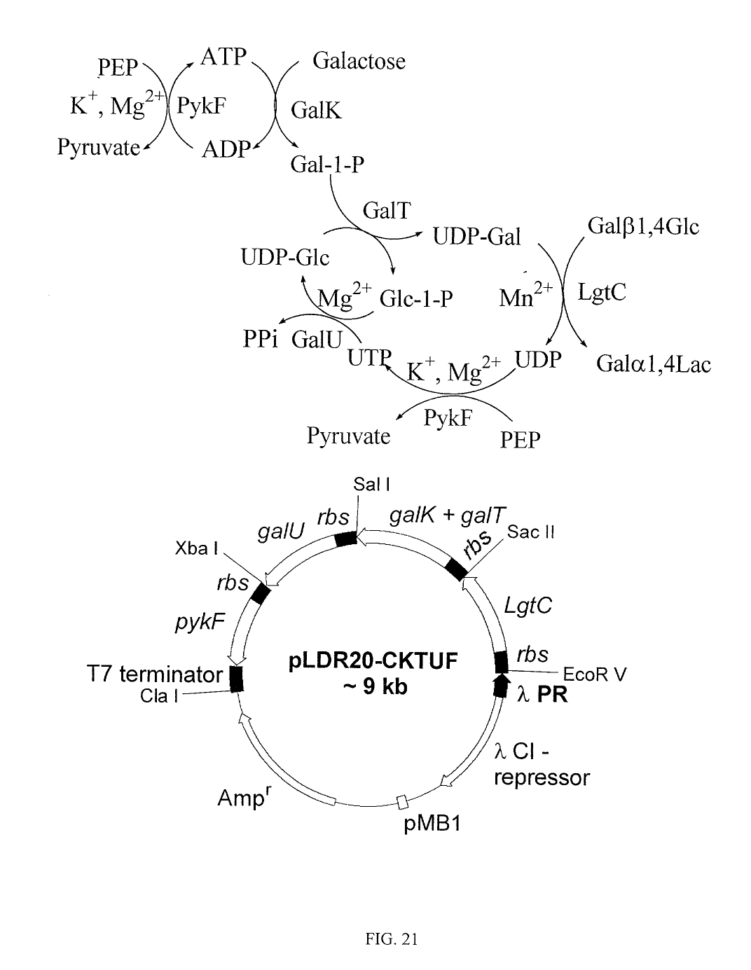

FIG. 22 Biosynthetic pathway and corresponding plasmid map for synthesis of globotriose using sucrose.

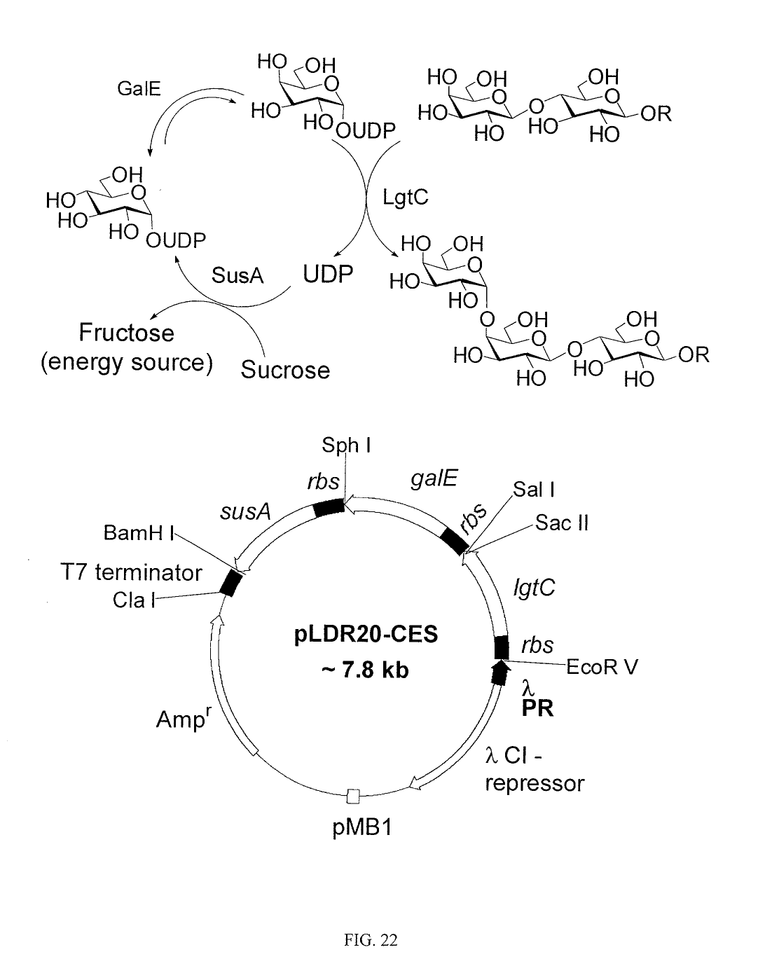

### DETAILED DESCRIPTION OF THE INVENTION

The present invention provides a method of producing glycoconjugates utilizing novel compositions and processes. In a preferred embodiment, a bioenergetic is provided to an organism containing a plasmid encoding at least one sugar-nucleotide-regenerating enzyme and at least one glycosyltransferase. The organism is able to utilize the bioenergetic to produce a sugar nucleotide. The sugar nucleotide produced is then utilized by the glycosyltransferase to add the sugar residue to an acceptor, thus producing a glycoconjugate. Preferably, the plasmid encodes enzymes to regenerate the sugar nucleotide from the by-product of the glycosyltransferase reaction (e.g., UDP).

Bioenergetics

An important aspect of the present invention is the ability to produce glycoconjugates using whole cells rather than individually isolating the biosynthetic enzymes and/or biochemical intermediates. Because biosynthesis of glycoconjugates requires energy, an energy source (bioenergetic) is provided to the organism of the present invention. As discussed in detail herein, essentially any bioenergetic that may be converted by the organism to produce a sugar nucleotide may be used. Furthermore, combinations of bioenergetics may be used.

One source of energy for organisms is saccharides. Examples of saccharides that may be used as bioenergetics in the present invention include monosaccharides, such as glucose, galactose, fructose, mannose, fucose; dissacharides, such as lactose or sucrose; or polysaccharides, such as starch. The saccharides are broken down to produce high-energy phosphate donors within the cell, such as ATP, PEP, UTP, GTP, and CTP. The resulting high-energy phosphate donors may be used by the organism to produce a sugar nucleotide.

Alternatively, the high-energy phosphate donor itself is provided directly to an organism of the present invention. Because the amount of high-energy phosphate donor produced by providing a saccharide to the organism is limited, directly providing a high-energy phosphate donor to the organism is preferred for large-scale production of glycoconjugates. In preferred embodiments, the high-energy phosphate donor is PEP or ATP. Internalization of such molecules into the cell can be facilitated by freeze thaw techniques or detergent treatment of the cells.

Examples of other preferred bioenergetics include polyphosphate, acetyl phosphate, and sucrose.

Although the biochemical pathways and their respective enzymes innate to the organism may be utilized in methods of the present invention, in preferred embodiments, heterologous genes encoding the enzymes are provided to the organism. By providing the genes heterologously, gene expression can be increased considerably over that of the innate gene. Furthermore, because expression of the innate genes is typically under strict control, i.e., negative feedback mechanisms, it is preferred that the heterologous gene is operably linked to a heterologous promoter.

Preferably, a heterologous gene is provided to the organism that is beneficial to the utilization of a given bioenergetic. For example, where PEP is the bioenergetic, pyruvate kinase may be provided to the organism. Pyruvate kinase uses PEP to convert nucleotide diphosphates (UDP, ADP) to nucleotide triphosphates (UTP, ATP). Examples of pyruvate kinases include PykF, PykA, yeast pyruvate kinase (Burke et al., J Biol Chem 1983, 258(4):2193-201), rat pyruvate kinase (Yamada et al., J Biol Chem 1990, 265(32):19885-91), and human pyruvate kinases (Zarza et al., Haematologica 2000, 85(3):227-32.; Takenaka et al., Eur J Biochem 1991, 198(1):101-6.; Harkins et al., Biochemistry 1977, 16(17):383 1-7).

Where ATP is the bioenergetic, nucleotide diphosphate kinase may be provided to the organism. Nucleotide diphosphate kinases, including NdK, from a variety of prokaryotic and eukaryotic sources are known in the art (e.g., Hama, H. et al., Gene 1991, 105, 31-36; Baker and Parker, FEMS Microbiol. Lett. 1994, 121, 293-296; Sundin et al., Mol Microbiol 1996, 20(5):965-79; Ulloa et al., Mol Biochem Parasitol 1995, 70(1-2): 119-29; Shimada et al., J Biol Chem 1993, 268(4):2583-9; Ishikawa et al., J Biol Chem 1992, 267(20):14366-72).

Where polyphosphate is the bioenergetic, polyphosphate kinase may be provided to the organism. Polyphosphate kinases, including PpK, from a variety of prokaryotic and eukaryotic sources are known in the art (e.g., Shiba, T. et al., Biochemistry (Mosc) 2000, 65, 315-323; Van Dien and Keasling, Biotechnol. Prog. 1999, 15, 587-593; Noguchi and Shiba, Biosci. Biotechnol Biochem. 1998, 62, 1594-1596; Trelstad et al., Appl Environ Microbiol 1999, 65(9):3780-6; Zago et al., Appl Environ Microbiol 1999, 65(5):2065-71; Tinsley et al., Infect Immun 1993, 61(9):3703-10; Robinson et al., Biochem Int 1984,8(6):757-69; J Gen Microbiol 1975, 88(1):65-74).

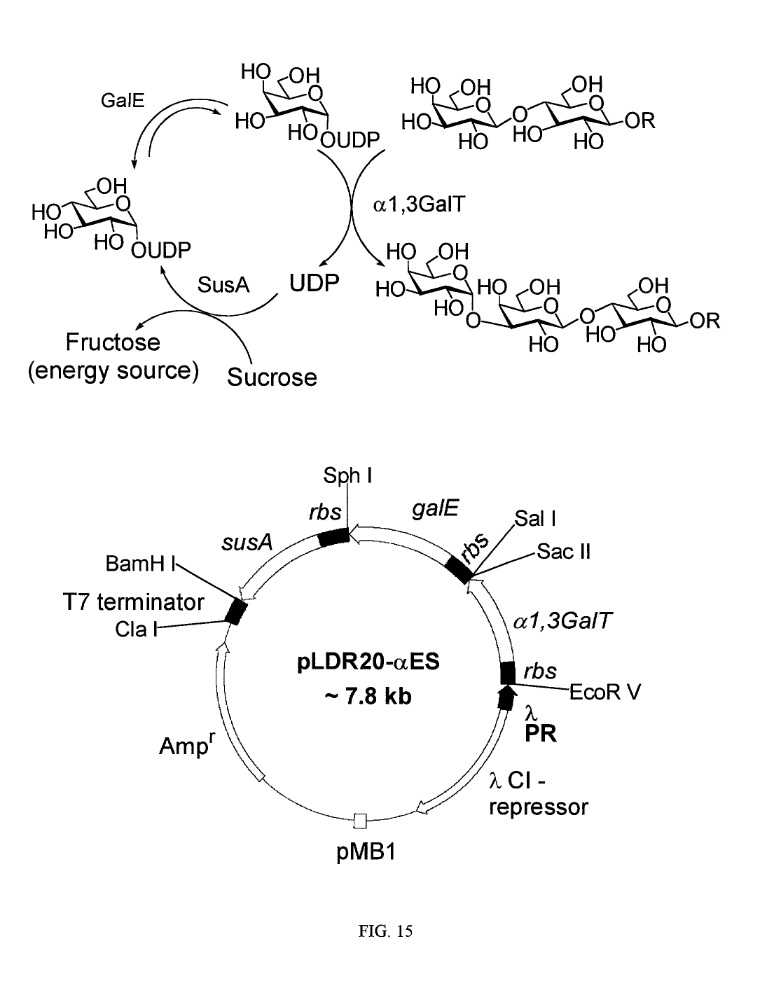

In certain embodiments, sucrose is used as the bioenergetic. Sucrose is a disaccharide consisting of fructose and glucose. Sucrose synthase (UDP-glucose: D-fructose 2-α-D-glucosyltransferase) catalyzes the synthesis and cleavage of sucrose. In some embodiments for the production of α-Gal, the regeneration of UDP-Gal utilizes only two enzymes, sucrose synthase (SS, EC 2.4.1.13) and UDP-Gal 4-Epimerase (GalE, EC 5.1.3.2). Using this UDP-Gal regeneration pathway, the α-Gal synthetic pathway may consist only of three enzymes (FIG. 15). The sucrose synthase is widespread in plant and has been well characterized. Unlike most enzymes of sugar-nucleotide metabolism, SS shows a wide specificity for the nucleoside base.

Sucrose synthase purified from rice grains, together with GalE and β1,4-galactosyltransferae, has been applied in the preparative synthesis of N-acetyllactosamine (LacNAc). A yield of 100% for 10 mM acceptor substrate was obtained under optimized conditions using a repetitive batch technique (Zervosen and Elling, J. Am. Chem. Soc. 1996, 118, 1836-1840). Combined with chemical method, UDP-N-acetyl-α-D-galactosamine has been obtained using purified sucrose synthase (Bulter et al., Carbohydr. Res. 1997, 305, 469-473). Plant recombinant SS has been obtained and applied in the gram-scale synthesis of ADP-glucose (Zervosen et al., J. Mol. Catalysis B: Enzymatic 1998, 5, 25-28).

The presence of SS has also been demonstrated in several species of green algae (e.g., Duran and Pontis, Mol. Cell Biochem. 1977, 16, 149-152; Salerno, Plant Sci. 1985, 42, 5-8; Salerno, Physiol. Plant 1985, 64, 259-264; Salerno et al., In: Pontis H. G.; Salerno, G. L.; Echeverria, E. J. (eds) Sucrose metabolism biochemistry, physiology and molecular biology, vol 14 (Current Topics in Plant Physiology: An American Society of Plant Physiologist Series), 1995, pp 34-39) and in extracts of Anabaena variabilis, a filamentous heterocystous cyanobacterium (e.g., Schilling and Ehrnsperger, Z. Naturforsch 1985, 40, 776-779). Also, two prokaryotic SS forms (SS-I and SS-II) were purified from Anabaena sp. strain PCC 7119. SS-II was biochemically characterized (Porchia et al., Planta 1999, 210, 34-40) and its gene sequence was reported to GenBank (Acc. # AJ010639). Anabaena SS II was shown to be a tetramer with each subunit having a molecular weight of 92-kDa. Sucrose synthase II exhibited optimal maximum activities between pH 7.5 and 8.2 in the sucrose-synthesis direction, and between 5.9 and 6.5 in the sucrose-cleavage direction. In the sucrose-synthesis direction, either Mg²⁺ or Mn²⁺ increased enzyme activity between 2- and 4-fold using UDP-Glc as substrates. However, the addition of Mn²⁺ strongly inhibits enzyme activity in the sucrose-cleavage direction, while Mg²⁺ has little effect. In the presence of uridine substrate (UDP-Glc or UDP), addition of ATP produces a strong inhibition in both directions.

Where O₂ is the bioenergetic, a series of enzymes for the utilization of O₂ to convert a nucleotide diphosphate to a nucleotide triphosphate may be used. For example, acetate kinase, inorganic pyrophosphatase, and pyruvate oxidase may be used together (Kim and Swartz, Biotech. Bioeng. 1999, 66, 180-188; Grabau, C. et al., J. Biol. Chem. 1989, 264, 12510-12519; Chang and Cronan, Biochemistry 1997, 36, 11564-11573. Wang, A. Y. et al., J. Biol. Chem. 1991, 266, 10959-10966.). Acetate kinases, including AcK (EC 2.7.2.1), from a variety of sources are known in the art (Alm et al., Nature 1999, 397(6715), 176-180; Kahane et al., J Bacteriol 1979, 137(2):764-72; Latimer and Ferry, J Bacteriol 1993, 175(21):6822-9).

Inorganic pyrophosphatases, including PPase (EC 3.6.1.1), from a number of sources may be used in embodiments of the present invention, including those of Heliobacter pylori (Oliva et al., Arch Microbiol 2000 174(1-2):104-110), Methanococcus jannaschii (Kuhn et al., Arch Biochem Biophys 2000 379(2):292-8), Bacillus subtilis (Shintani et al., FEBS Lett 1998 439(3):263-6; Young et al., Microbiology 1998 144 (Pt 9):2563-71), human (Fairchild et al., Biochim Biophys Acta 1999 1447(2-3):133-6; Baykov et al., Prog Mol Subcell Biol 1999 23:127-50), yeast (Pohjanjoki et al., Biochemistry 1998 37(7):1754-61; Kolakowski et al., Nucleic Acids Res 1988 16(22):10441-52; Heikinheimo et al., Eur J Biochem 1996 239(l):138-43), bovine (Yang and Wensel, J Biol Chem 1992 267(34):24641-7), and plant (Maeshima, Biochim Biophys Acta 2000 1465(1-2):37-51; Rodrigues, Mol Cell Biol 1999 19(11):7712-23; Suzuki et al., Plant Cell Physiol 1999 40(8):900-4).

Pyruvate oxidases, including PoxB (EC 1.2.3.3), from sources such as Lactobacillus plantarum and Pediococcus sp. may be used in certain embodiments of the present invention.

Sugar-Nucleotide Producing Enzymes

The bioenergetic is utilized by the organism to produce a sugar nucleotide. This sugar nucleotide donor is then used by a glycosyltransferase to add the sugar moiety to a saccharide. In preferred embodiments, a precursor is provided to the organism. An enzyme recognizes the precursor and attaches it to a nucleotide to create the sugar nucleotide. Of course, in certain embodiments, a non-nucleotide donor molecule may be provided to the organism for use by the glycosyltransferase (Lougheed et al., J Biol Chem 1999, 274(53):37717-22.). When the donor is a sugar nucleotide, the end product of the glycosyltransferase reaction is a nucleotide diphosphate or a nucleotide monophosphate. In preferred embodiments, the organism is engineered to efficiently regenerate the sugar-nucleotide, thus, continually producing more sugar-nucleotide for the glycosyltransferase reaction.

An important aspect of the present invention is the ability to tailor the compositions and methods to specific sugar nucleotides. Genes encoding enzymes involved in sugar-nucleotide generation and regeneration are known in the art (e.g., EP 0870841, incorporated herein by reference in its entirety). In light of the present invention, one of ordinary skill in the art could utilize these genes to customize the compositions and methods to a given sugar nucleotide as discussed below. In preferred embodiments, the one or more sugar-nucleotide regeneration genes are over-expressed by the organism.

Examples of sugar nucleotides that may be regenerated include UDP-Gal, UDP-Glc, UDP-GlcNAc, UDP-GalNAc, UDP-GlcA, CMP-NeuNAc, GDP-Man, GDP-Fuc, and UDP-GalA.

A. UDP-Gal

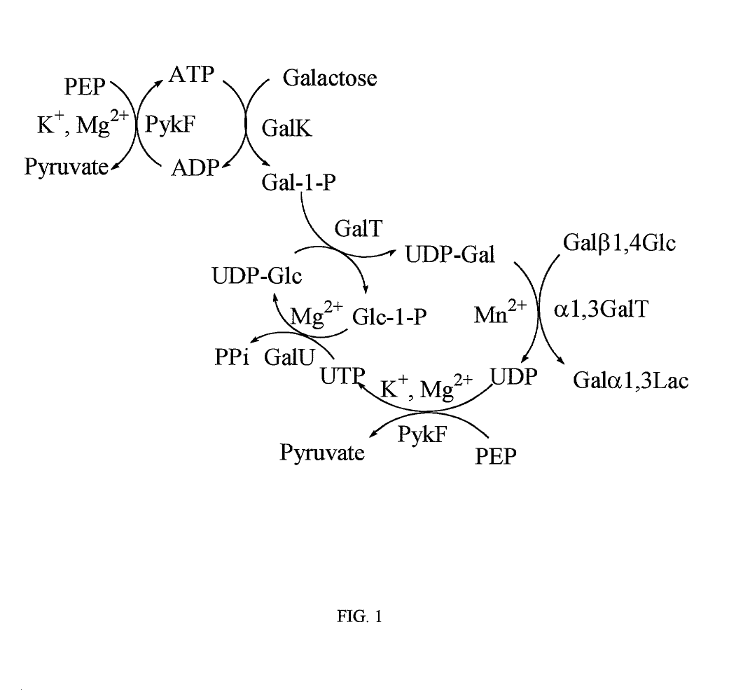

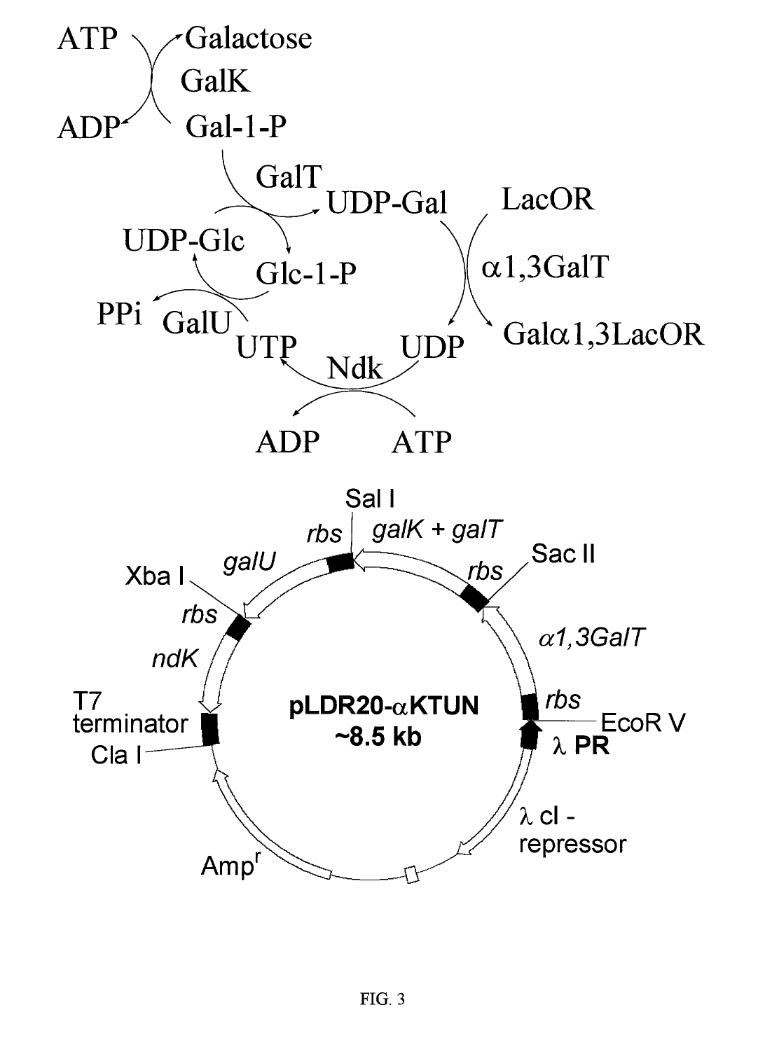

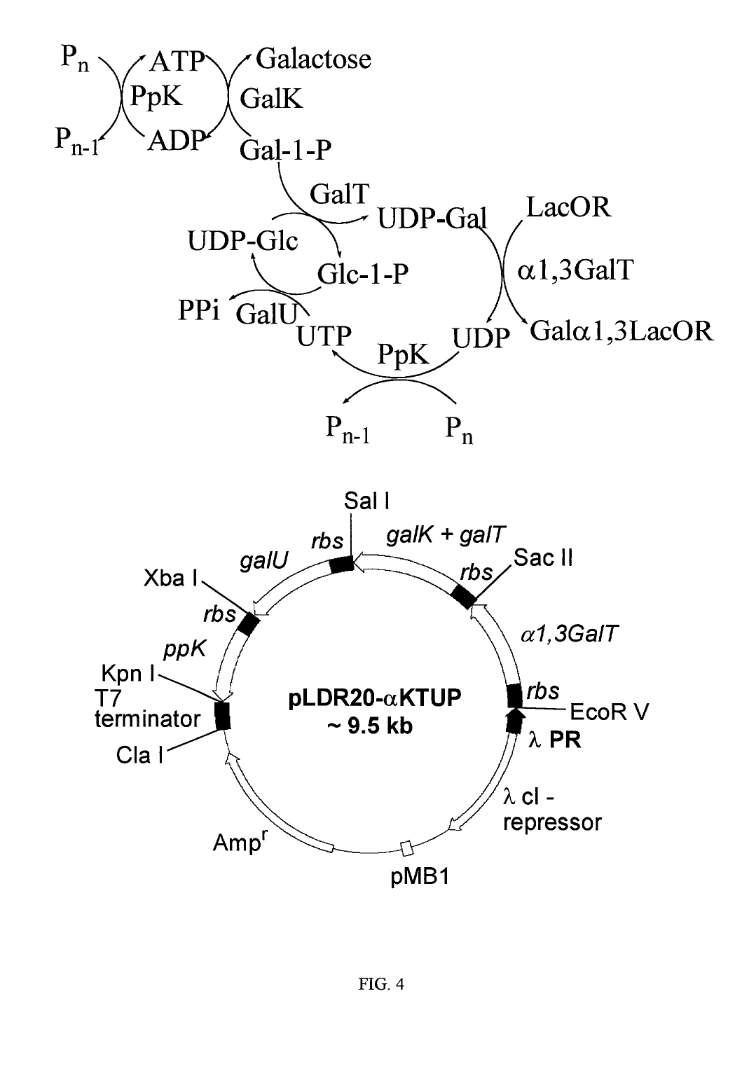

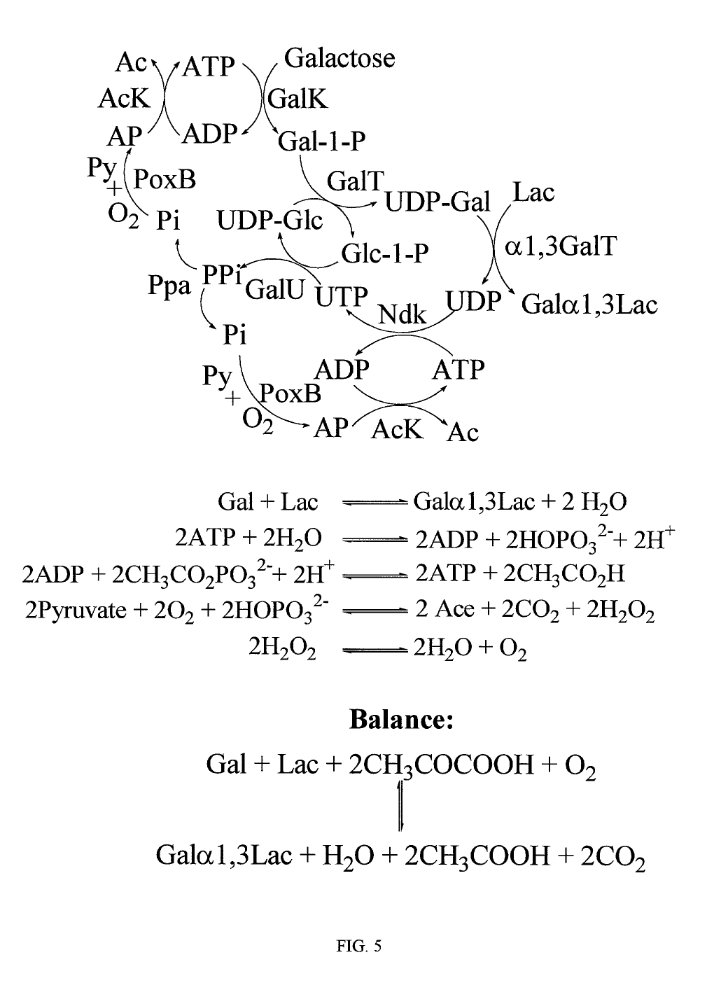

In certain embodiments, UDP-Gal is regenerated. In such embodiments, the precursor galactose is provided to the organism. Galactose is converted into Gal-1-P, which is subsequently converted to UDP-Gal. After the Gal of UDP-Gal is utilized by the glycosyltransferase, the resulting UDP is converted into UTP, which is subsequently converted into UDP-Glc. The UDP-Glc is then used to create UDP-Gal. Examples of enzymes used together to regenerate UDP-Gal include GalK, GalT, and GalU. How these enzymes, along with bioenergetics and glycosyltransferase, complete the above tasks is exemplified in FIG. 1, FIG. 3, FIG. 4, FIG. 5 and FIG. 15.

Other enzymes may be used to regenerate UDP-Gal. For example, in certain embodiments wherein UDP-Gal is regenerated, an epimerase, such as GalE, is included with GLK, PGM, and GalU. In other embodiments wherein UDP-Gal is regenerated, a sucrose synthase and GalE are used in combination as shown in FIG. 15.

B. UDP-Glc

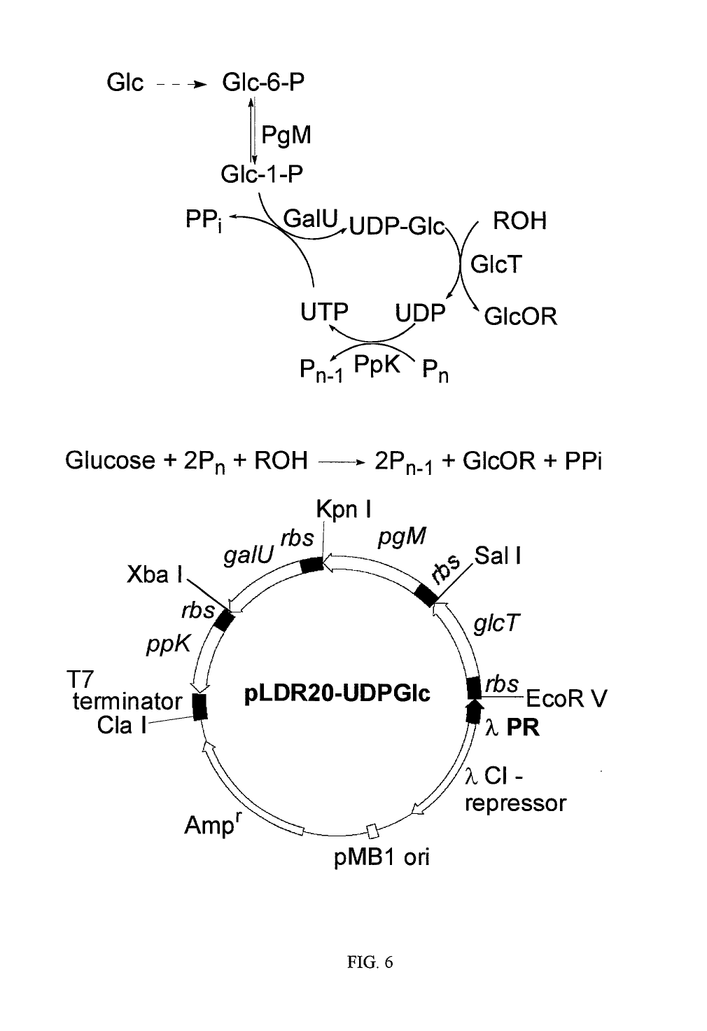

In other embodiments, UDP-Glc is regenerated. One method of regenerating UDP-Glc is through the glucose metabolism pathway as diagrammed in FIG. 6. UDP-Glc is regenerated by the combination of phosphoglucomutase (PgM, EC 5.4.2.2) (Leyva-Vazquez and Setlow, J. Bacteriol. 1994, 176, 3903-3910; Lu and Kleckner, Bacteriol. 1994, 176, 5847-5851; Pradel and Boquet, Res. Microbiol. 1991, 142, 37-45), glucose-1-phosphate uridyltransferase (GalU) and polyphosphate kinase (PpK). Since glucose is phosphorylated during the uptake into E. coli, glucokinase (GlK, EC 2.7.1.2) (Skarlatos and Dahl, J. Bacteriol. 1998, 180, 3222-3226; Meyer, D. et al., J. Bacteriol 1997, 179, 1298-1306; Arora and Pedersen, Arch Biochem. Biophys. 1995, 319, 574-578) is not required when the organism is E. coli.

Other enzymes may be used to regenerate UDP-Glc. For example, in certain embodiments for regerating UDP-Glc, an epimerase, such as GalE, is included with GalU, GalT, and GalK.

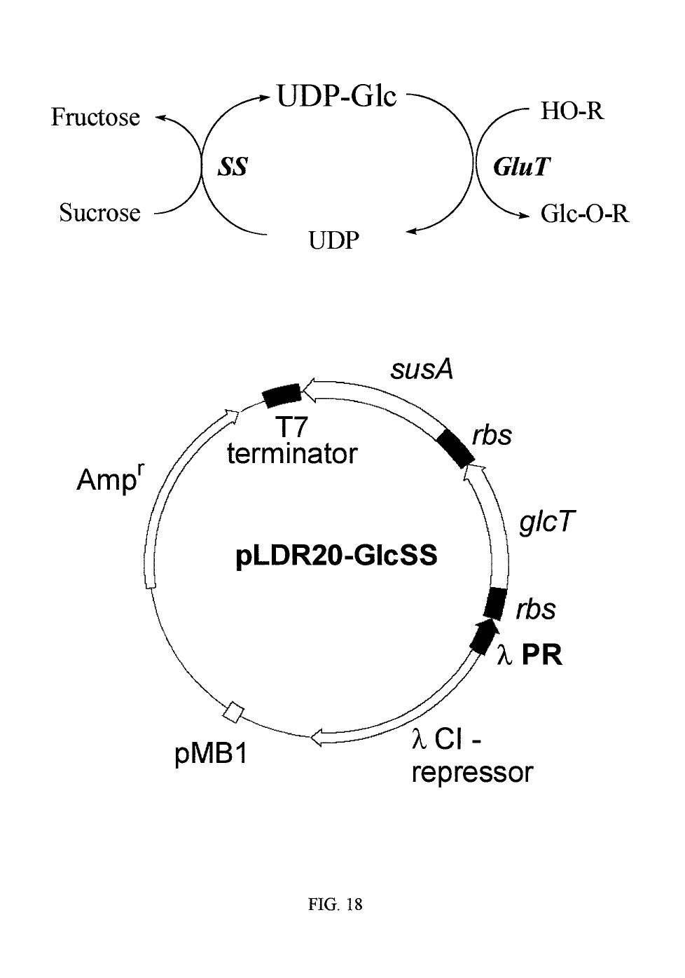

Another example uses a sucrose synthase, which converts UDP to UDP-Glc directly with the consumption of sucrose (FIG. 18). In this embodiment, only two enzymes (sucrose synthase and a glucosyltransferase) complete the reaction. Furthermore, the fructose produced by the sucrose cleavage reaction can be used as a nutrient for the cell. Thus, in this embodiment, sucrose serves both as a bioenergetic and a source of glucose.

C. UDP-GlcNAc

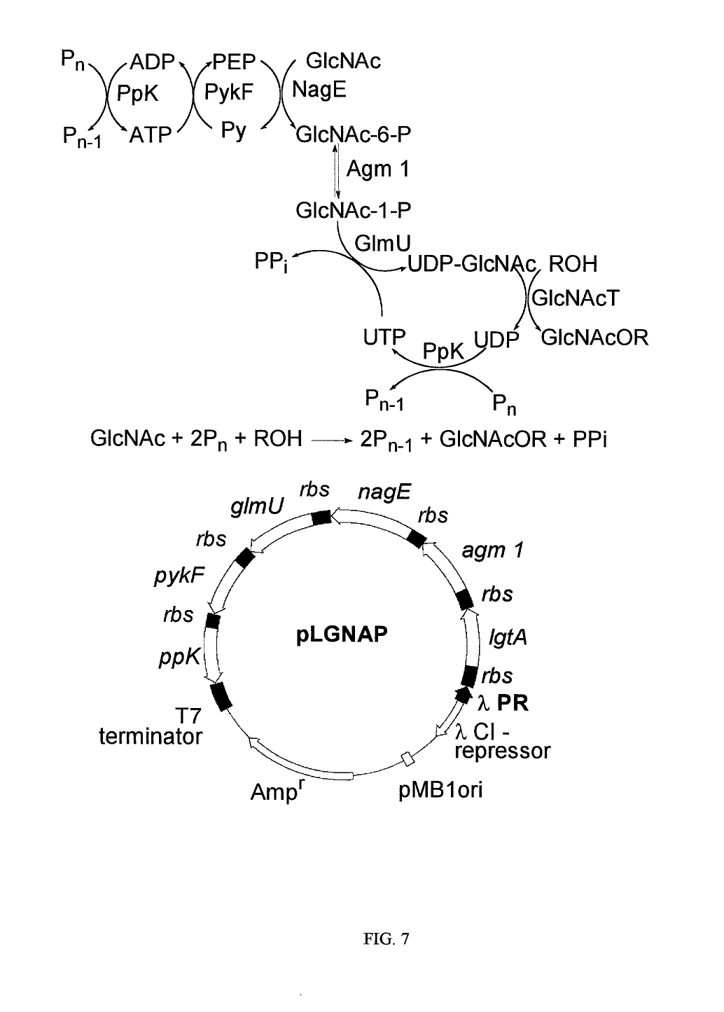

In other embodiments, UDP-GlcNAc is regenerated. One method of regenerating UDP-GlcNAc is diagrammed in FIG. 7. In this UDP-GlcNAc regeneration system, GlcNAc-1-phosphate uridyltransferase from Escherichia coli (glmU; Brown, K. et al., EMBO J. 1999, 18, 4096-4107; Gehring, A. M. et al., Biochemistry 1996, 35, 579-585; Mengin-Lecreulx and van Heijenoort, J. Bacteriol. 1993, 175, 6150-6157), N-acetylglucosamine permease from Vibrio furnissii (nagE, Yamano, N. et al., Biosci. Biotechnol. Biochem. 1997, 61, 1349-1353), N-acetylglucosamine-phosphate mutase from Saccharomyces cerevisiae (agml; Mio, T. et al., J. Biol. Chem. 1999, 274, 424-429) are used to regenerate the sugar nucleotide. Also, used in this system are the gycosyltransferase β1,3GlcNAc transferase from Neisseria meningitidis (LgtA; Blixt, O. et al., Glycobiology 1999, 9, 1061-1071) together with polyphosphate kinase and pyruvate kinase (ppK and pykF, respectively). In the system shown in FIG. 7, polyphosphate is the bioenergetic.

In a preferred embodiment, all of the genes of the UDP-GlcNAc regeneration system diagrammed in FIG. 7 are cloned into a cassette to produce the plasmid pLGNAP. This vector includes genes (ppK, pykF, glmU, nagE, and agm1) to regenerate UDP-GlcNAc from UDP using pyrophosphate as a bioenergetic and a glycosyltransferase (lgtA) that transfers the sugar from the sugar nucleotide to an acceptor molecule. This plasmid (FIG. 7), when transfected into E. coli, is particularly useful for UDP-GlcNAc regeneration and GlcNAcβ1,3LacOR synthesis. GlcNAcβ1,3LacOR is a core structure in α-Gal pentasaccharides (important for xenotransplantation research) and lipopolysaccharides on the membrane of Neisseria meningitidis.

D. UDP-GalNAc

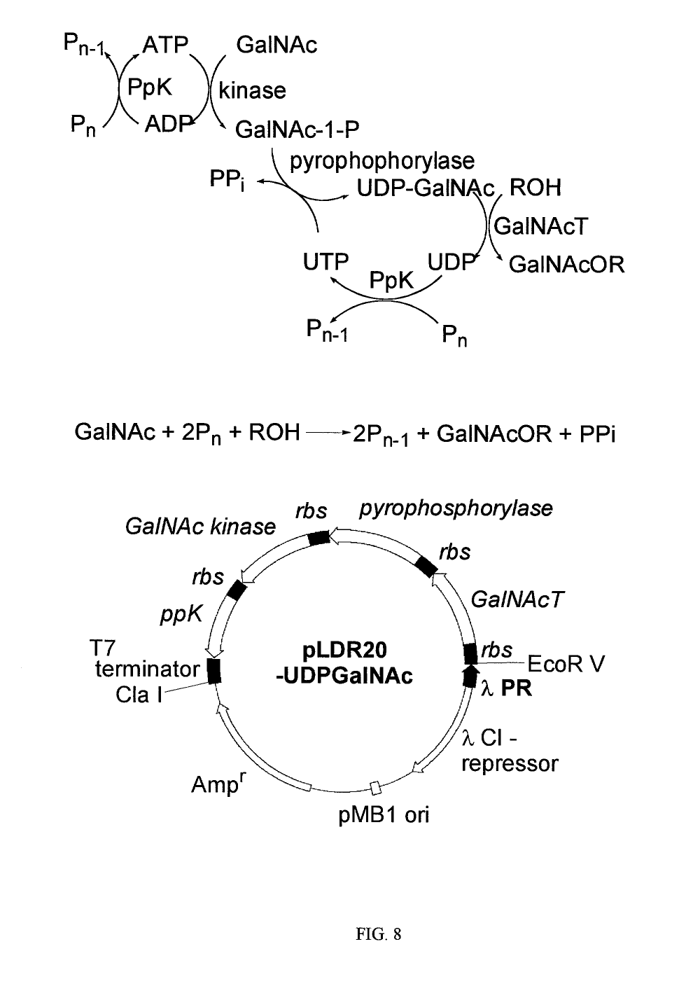

In other embodiments, UDP-GalNAc is regenerated. One method of regenerating UDP-GalNAc is diagrammed in FIG. 8. In this method, UDP-GalNAc is biosynthesized directly from GalNAc by a GalNAc-1 kinase and then by a pyrophosphorylase (uridyltransferase). This route is derived from a pathway for salvage of GalNAc generated by the degradation of glycosaminoglycans and glycoproteins in eukaryotes. One utility of UDP-GalNAc regeneration is that it can be coupled with a human UDP-GalNAc:2′-fucosylgalactoside-α-3-N-acetylgalactosaminyl transferase to synthesize human blood type A antigen.

The first gene in the pathway, GalNAc-1-phosphate kinase, was first identified by Pastuszak and co-workers from pig kidney in 1996 (Pastuszak, I. et al., J. Biol. Chem. 1996, 271, 20776-20782). The enzyme shows high specificity for GalNAc over other N-acetylated and non-acetylated aminosugars. It is a monomeric, 50 kDa protein with a divalent metal requirement (5 mM Mg²⁺ optimal). The enzyme is most active with ATP as the high-energy phosphate donor. However, some activity is also detected with ITP, acetyl-phosphate and phosphoenolpyruvate (PEP). Significant GalNAc-1-P kinase activity has also been detected in human kidney and liver and the sequence of peptides from the GalNAc kinase have been reported (Pastuszak, I. et al., J. Biol. Chem. 1996, 271, 23653-23656). These peptides showed very high homology with the human galactokinase reported on chromosome 15 and in fact the authors, based on further biochemical evidence, reassigned this human kinase as GalNAc kinase.

The second enzyme of this pathway is UDP-GalNAc pyrophosphorylase. Purified to homogeneity by Szumilo and others (Szumilo, T. et al., J. Biol. Chem. 1996, 271, 13147-13154; Wang-Gillam, A. et al., J. Biol. Chem. 1998, 273, 27055-20757), the protein is 64 kDa by SDS-PAGE. The Kₘ value for GalNAc-1-P was 0.29 mM and for GlcNAc-1-P was 1.1 mM. This indicates that at low concentrations, UDP-GlcNAc is the preferred substrate. However, at 5 mM, UDP-GalNAc is as effective as UDP-GlcNAc. The enzyme's pH optimum is between 8.5 and 8.9 and it requires a divalent metal for activity (Mn²⁺>Mg²⁺>Co²⁺).

As in the case of UDP-Gal and UDP-Glc co-regeneration organisms, UDP-GalNAc can also be biosynthesized from UDP-GlcNAc by an epimerase UDP-GlcNAc 4-epimerase (EC 5.1.3.7). The protein with this activity has been isolated from both prokaryotic and eukaryotic sources. Creuzenet and co-workers identified the wbpP gene encoding the UDP-GlcNAc 4-epimerase activity from A. aeruginosa (Creuzenet, C. et al., J. Biol. Chem. 2000, 275, 19060-19067). The proposed gene product shows a conserved nucleotide-binding-protein motif (GXXGXXG; SEQ ID NO: 1) and a catalytic triad (SYK) with E. coli UDP-Gal 4-epimerase (GalE), which provides an opportunity to identify and clone enzymes with this function from other prokaryotic sources based on sequence alignment and other bioinformatic methods.

E. UDP-GlcA

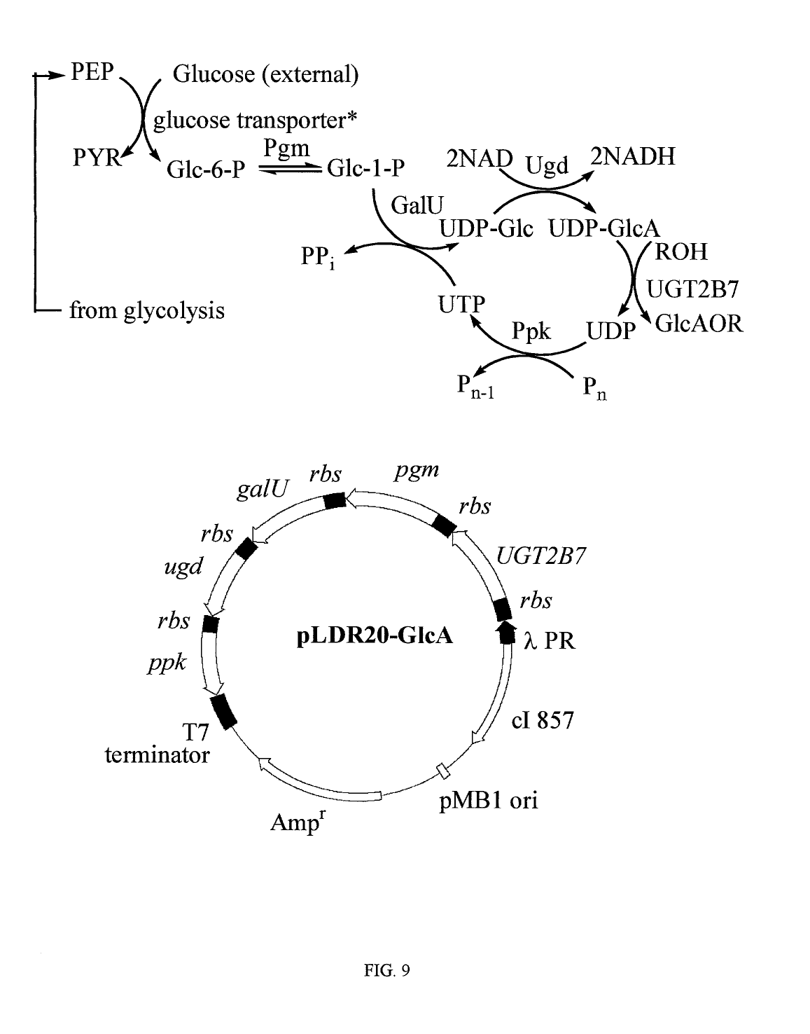

In other embodiments, UDP-GlcA is regenerated. One method of regenerating UDP-GlcA is diagrammed in FIG. 9. In all living systems, UDP-GlcA is synthesized from UDP-Glc by UDP-Glc 6-dehydrogenase (UDPGDH). This step is the control point to all the subsequent UDP-GlcA utilizing reactions. One equivalent of UDP-Glc is oxidized to one equivalent of UDP-GlcA with concomitant reduction of two equivalents of NAD⁺ to NADH. The UDP-GlcA regeneration system may be constructed by adding the ugd gene encoding UDP-GlcA 6-dehydrogenase and substituting the gene of glucosyltransferase with a human UGT2B7 gene into the UDP-Glc regeneration system described in FIG. 6 to produce pLDR20-GlcA (See FIG. 9). An advantage of this system is that the NAD⁺ co-factor is provided by normal cellular metabolism.

UDP-Glc 6-dehydrogenase activity has been isolated from variety of organisms. The enzyme has been cloned from a number of sources including both human and mouse (Spicer, A. P. et al., J. Biol. Chem. 1998, 273, 25117-25124), bovine kidney (Lind, T. et al., Glycobiology 1999, 9, 595-600), and prokaryotic organisms Sinorhizobium meliloti (Kereszt, A. et al., J. Bacteriol. 1998, 180, 5426-5431), E. coli K5 (De Luca, C. et al., Bioorg. Med. Chem. 1996, 4, 131-134), and Bacillus subtilis 168 (Pagni, M. et al., Microbiology 1999, 145, 1049-1053.). The gene for this enzyme is also present in the Chlorella virus PBCV-1 and has been found to produce a functional protein early in the infection (Landsterin, D. et al., Virology 1998, 250, 388-396).

Although essentially any of these UDP-Glc 6-dehydrogenases may be used in the compositions and methods of the present invention, a preferred UDP-Glc 6-dehydrogenase is encoded by the ugd gene from E. coli K5 (43 kD)( De Luca et al., Bioorg Med Chem 1996, 4(1):131-41) because it does not contain internal restriction sites for the other enzymes used in the construction of the multiple enzyme vectors. This property greatly facilitates the construction of the plasmid.

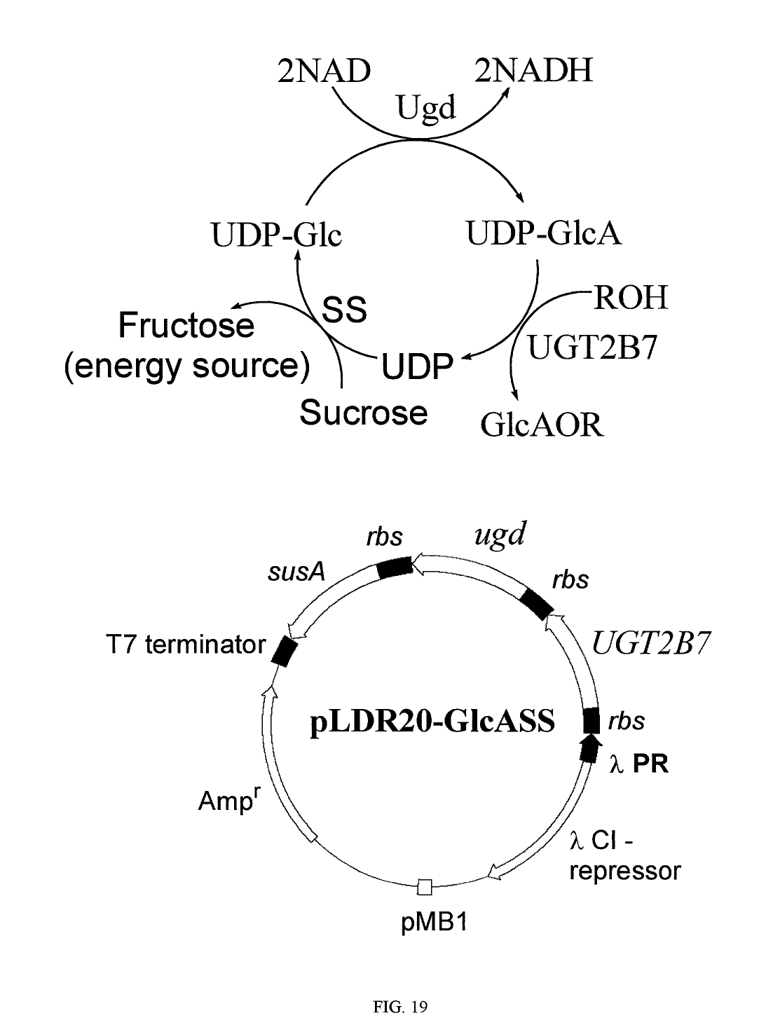

As is the case with UDP-Glc, UDP-Glc-A can be regenerated using sucrose synthetase and UDP-Glc 6-dehydrogenase as shown in FIG. 19.

F. CMP-NeuNAc

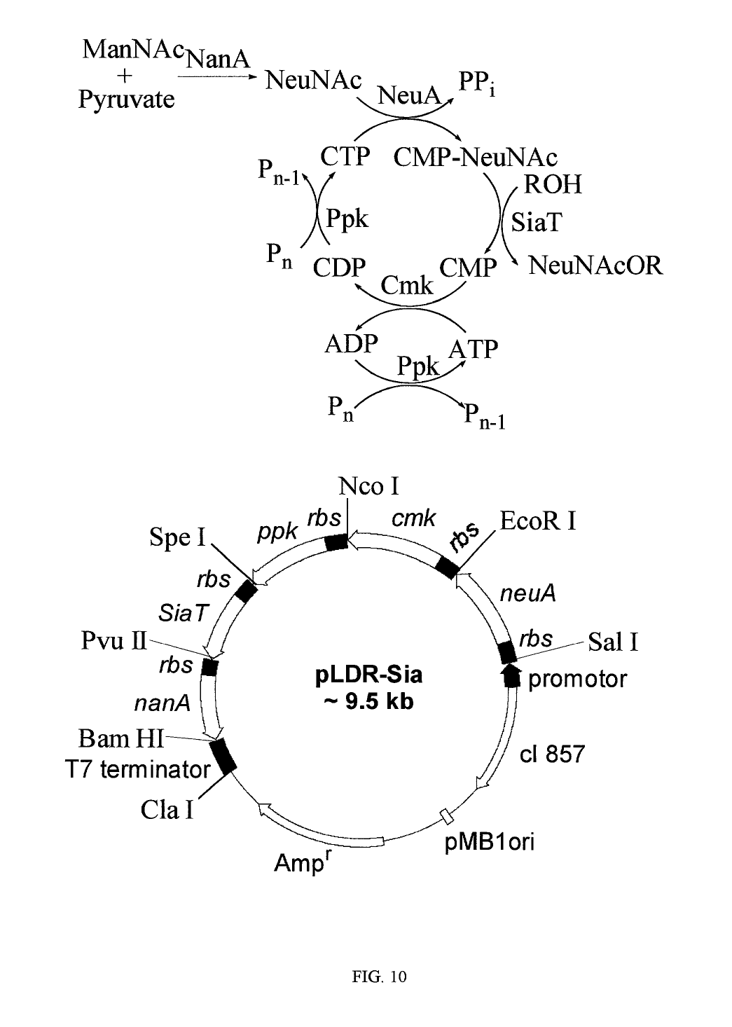

In other embodiments, CMP-NeuNAc is regenerated. One method of regenerating CMP-NeuNAc is diagrammed in FIG. 10. A particularly useful plasmid for the regeneration of CMP-NeuNAc is pLDR-Sia (FIG. 10). This plasmid encodes sialic acid aldolase (NanA), CMP-Neu NAC synthase (NeuA), CMP kinase (Cmk), and polyphosphate kinase (Ppk) along with the glycosyltransferase α2,3 (or α2,6)-sialyltransferase (SiaT).

NeuAc aldolase (NanA, N-acetylneuraminate lyase, EC 4.1.3.3) catalyzes the reversible cleavage of NeuAc to form pyruvate and ManNAc. The enzyme has been exploited for the synthesis of NeuNAc or its derivatives (Murakami, M. et al., Carbohydr. Res. 1996, 280, 101-110; Mahmoudian, M. et al., Enzyme. Microb. Technol. 1997, 20, 393-400; Maru, I. et al., Carbohydr. Res, 1998, 306, 575-578; Aisaka, K. et al., Biochem. J. 1991, 276, 541-546; Walters, D. M. et al., J. Bacteriol. 1999, 181, 4526-4532; Lilley, G. G. et al., Protein Expr. Purif 1992, 3, 434-440). The E. coli NanA is a tetramer with an optimum pH around 7.7. The Kₘ for NeuNAc is 4.3 mM and pyruvate competitively inhibits the cleavage reaction. The enzyme belongs to the Schiff-base-forming Class I aldolases and X-ray crystallographic structure available (Aisaka, K. et al., Biochem. J. 1991, 276, 541-546; Uchida, Y. et al., J. Biochem. (Tokyo) 1984, 96, 507-522. When the E. coli gene encoding NanA was cloned into the pET15b vector downstream of the T7 promoter, the overexpressed protein consisted of more than 50% of the total cellular protein. About 30,000 units of active enzyme can be obtained from one liter of bacterial culture.

CMP-NeuNAc synthetase (NeuNAcS, N-Acetylneuraminic acid cytidylyltransferase, EC 2.7.7.43) catalyzes the formation of CMP-NeuNAc (Vann, W. F. et al., J. Biol Chem. 1987, 262, 17556-17562; Vionnet, J. et al., Glycobiology 1999, 9, 481-487; Munster, A. K. et al., Proc. Natl. Acad. Sci U.S.A. 1998, 95, 9140-9145). The enzyme purified from E. coli K1 requires Mg²⁺ or Mn²⁺ and exhibits optimal activity between pH 9.0 and 10. The apparent Kₘ for CTP and NeuNAc are 0.31 and 4 mM, respectively. The gene encoding NeuNAcS from E. coli serotype 07 K1 was isolated and overexpressed in E. coli W3110 with expression level up to 8-10% of the soluble E. coli protein. The over-expressed synthetase was purified to greater than 95% homogeneity and used directly for the synthesis of CMP-NeuNAc and derivatives (Shames, S. L. et al., Glycobiology 1991, 1, 187-191). Other researchers have also reported the enzymatic synthesis of CMP-NeuNAc using NeuNAcS (Aisaka, K. et al., Biochem. J. 1991, 276, 541-546; Kittelmann, M. et al., Appl. Microbiol. Biotechnol. 1995, 44, 59-67).

CMP kinase from E. coli is a monomeric protein of 225 amino acid residues. The protein exhibits little overall sequence similarity to other known NMP kinases. However, the residues involved in substrate binding and/or catalytic motif(s) were found to be conserved, and sequence comparison suggests a similar global structure found in adenylate kinases or several other CMP/UMP kinases (Bucurenci, N. et al., J. Biol. Chem. 1996, 271, 2856-2862; Briozzo, P. et al., Structure 1998, 6, 1517-1527). Substrate specificity studies show that CMP kinase from E. coli is active with ATP, dATP, or GTP as donors and with CMP, dCMP, and arabinofuranosyl-CMP as acceptors (Bucurenci, N. et al., J. Biol. Chem. 1996, 271, 2856-2862; Briozzo, P. et al., Structure 1998, 6, 1517-1527).

G. GDP-Man

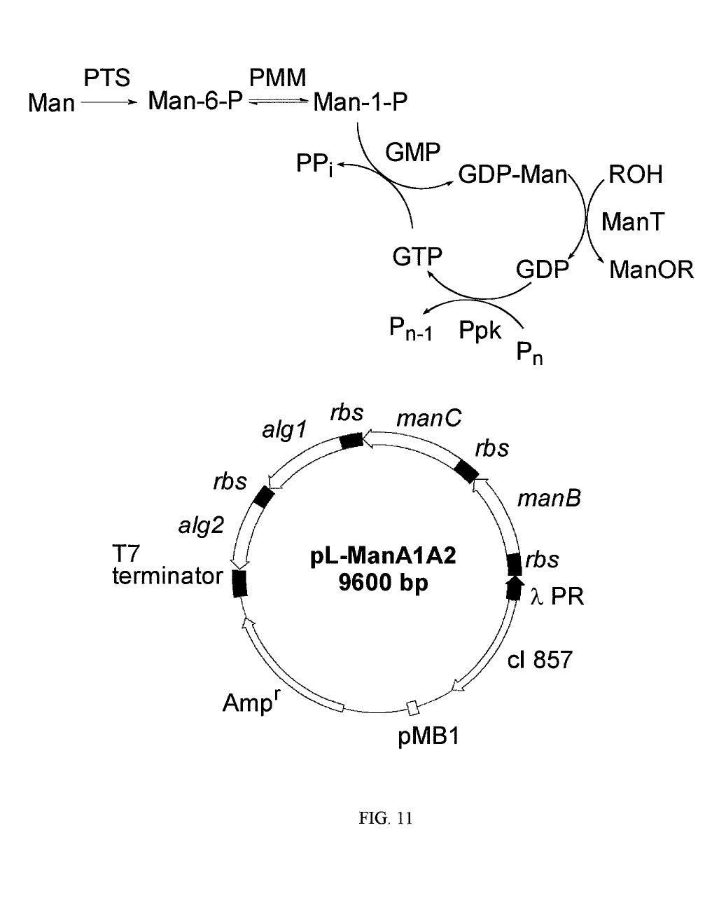

In other embodiments, GDP-Man is regenerated. One method of regenerating GDP-Man is diagrammed in FIG. 11. A particularly useful plasmid for the regeneration of GDP-Man is pL-ManA1A2 (FIG. 11).

The biosynthesis of GDP-mannose can start with mannose 6-phosphate which is automatically phosphorylated from mannose when transported into the cell via the PEP-dependerit transporter system (PTS). In a preferred embodiment, phosphomannomutase (PMM, EC 5.4.2.8) and GDP-mannose pyrophosphorylase (GMP, EC 2.7.7.13), two key enzymes contributing to the pathway of GDP-mannose regeneration, are overexpressed along with a mannosyltransferase for the synthesis of mannose-terminated glycoconjugates (FIG. 11).

PMM catalyzes the interconversion of mannose-6-phosphate and mannose-1-phosphate. In the rfb gene cluster of E. coli 09 strain, rfbK was indicated to encode PMM and rfbM encodes GDP-mannose pyrophosphorylase (GMP, EC 2.7.7.13)(Marolda and Valvano, J. Bacteriol. 1993, 175, 148-158; Sugiyama, T. et al., Microbiology 1994, 140, 59-71; Jayaratne, P. et al., J. Bacteriol. 1994, 176, 3126-3139). In E. coli K-12 strain, there is a wca (cps) gene cluster comprising another pair of isogenes termed cpsG(manS) and cpsB(manC) encoding PMM and GMP, respectively. The cpsG(manS) and cpsB(manC) genes contribute to the production of both GDP-mannose and GDP-fucose (Aoyama, K. et al., Mol. Biol. Evol. 1994, 11, 829-838). The manB gene (1371 bp) encodes a predicted 50.5 kDa protein that requires Mg²⁺ or Mn²⁺ for activity (Zielinski, N. A. et al., J. Biol. Chem. 1991, 266, 9754-9763; Goldberg, J. B. et al., J. Bacteriol. 1993, 175, 1605-1611; Coyne, M. J. et al., J. Bacteriol. 1994, 176, 3500-3507; Ye, R. W. et al., J. Bacteriol. 1994, 176, 4851-4857). The crystal structure of the enzyme has been published (Regni, C. A. et al., Acta Crystallogr. D. Biol. Clystallogr. 2000, 56, 761-762). The manC gene has 1437 bp encoding a 53.0 kDa protein which is also termed GTP:mannose 1-phosphate guanylyltransferase (EC 2.7.7.22) describing the reverse reaction.

H. GDP-Fuc

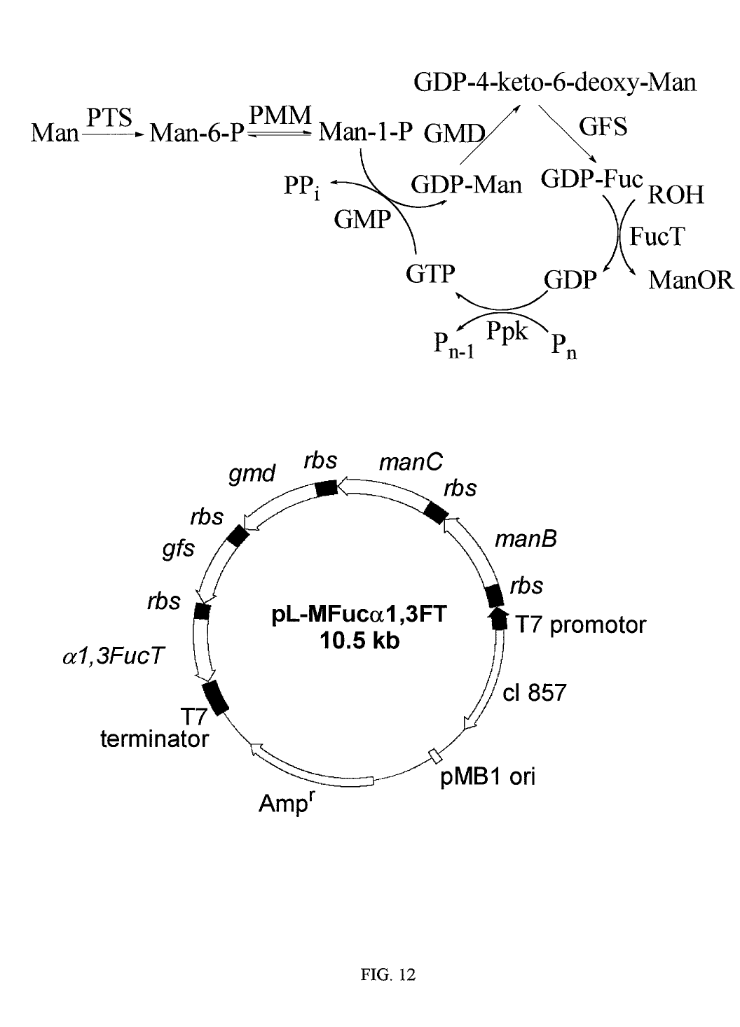

In other embodiments, GDP-Fuc is regenerated. One method of regenerating GDP-Fuc is diagrammed in FIG. 12. A particularly useful plasmid for the regeneration of GDP-Fuc is pL-Fucα1,3FT (FIG. 12).

The major pathway to generate GDP-fucose from GDP-mannose is present in both prokaryotes and eukaryotes. Two routes can be proposed. One of the routes carries out the formation of GDP-fucose from GDP-mannose in three steps but two enzymes. An alternate pathway is a two step procedure to form GDP-fucose from fucose (Pastuszak, I. et al., J. Biol. Chem. 1998, 273, 30165-30174). The first route is illustrated in FIG. 12. A GDP-Fuc regenerating organism can be easily obtained by modifying an existing GDP-Man regenerating organism by adding genes that encode GMD (GDP-D-mannose 4,6-dehydratase, EC 4.2.1.47) and a bifunctional GFS (GDP-L-fucose synthetase) or GMER (GDP-4-keto-6-deoxy-D-mannose epimerase/reductase) and substituting the mannosyltransferase with a fucosyltransferase such as α1,3FucT (FIG. 12).

Three steps are involved in the conversion of GDP-fucose from GDP-mannose including 4,6-dehydrogenation, 3,5-epimerization, and 4-reduction (Bonin, C. P. et al., Proc. Natl. Acad. Sci. USA 1997, 94, 2085-2090; Ohyama, C. et al., J. Biol Chem. 1998, 273, 14582-14587). The enzyme involved in the first step, GMD from E. coli, has been cloned, expressed and characterized (Mattila et al., Glycobiology 2000 10(10): 1041-7; Andrianopoulos et al., J Bacteriol 1998 180(4):998-1001; Sturla et al., FEBS Lett 1997 412(l):126-30). Metal ion Ca²⁺ and Mg²⁺ are required for the enzyme activity (Sturla et al., 1997). The gmd gene may be cloned by PCR from the wca (cps) gene cluster of E. coli K-1 2, which contains 1122 bp encoding a predicted 42.1 kDa protein (Stevenson et al., J Bacteriol 1996 178(16):4885-93).

E. coli protein GSF displays dual 3,5-epimerase and 4-reductase activities. Both epimerization and reduction reactions occur at the same site within a Ser-Tyr-Lys catalytic triad. The gene gfs (966 bp) found in E. coli K-12 encodes a 36.1 kDa protein (Rizzi, M. et al., Structure 1998, 6, 1453-1465).

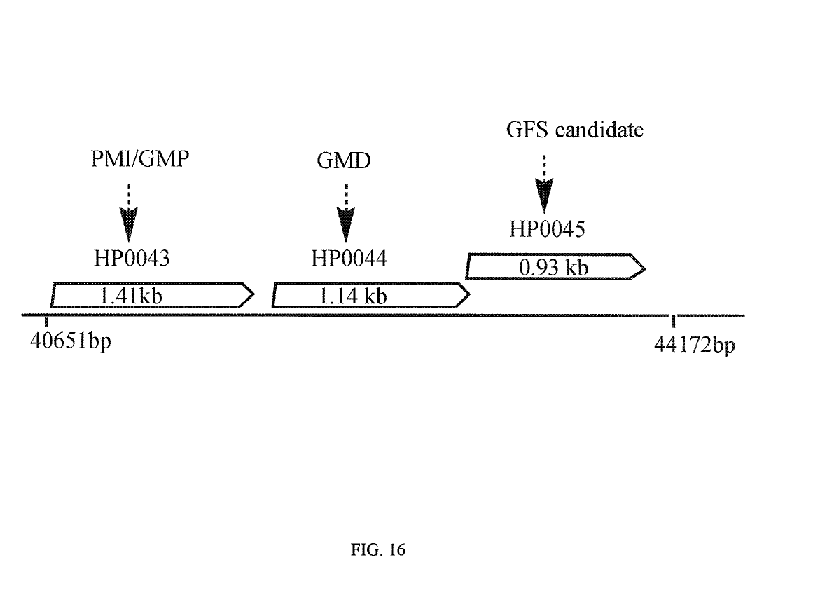

Upon examination of the genomic database of Helicobactor pylori (ATCC strain NO.700392), the inventors have identified four important enzymes (PMI,GMP,GMD,GFS) in the biosynthesis of GDP-Fuc in H. pylori that are encoded by a gene cluster. H. pylori can mimic the host surface antigens to escape the elimination by the host immune system. For example, LPS O-antigen of H. pylori commonly expresses human oncofetal antigens Lewis X and Lewis Y. Several fucoslytransferases have been identified and cloned from H. pylori (Martin et al., J. Biol. Chem. 1997, 272, 21349-21356; Wang et al., Mol. Microbiol. 1999, 31, 1265-1274; Rasko et al., J. Biol. Chem. 2000, 275, 4988-4994; Alm et al., Nature 1999, 397, 176-180; Wang et al., Microbiology 1999, 145, 3245-3253; Ge et al., J. Biol. Chem. 1997, 272, 21357-21363), however the source of donor GDP-fucose has not been previously determined. A BLAST gene search against the genome of H. pylori using sequences of GDP-fucose biosynthesis enzymes revealed a gene cluster (40651nt to 44172nt, HP0043, HP0044, HP0045 in FIG. 16) putatively responsible for GDP-fucose biosynthesis.

It is common that genes for the synthesis of certain sugar nucleotides are generally clustered together within bacterial genomes. HP0043 and HP0044 have been identified as putative PMI/GMP and GMD. In the genomic database, HP0045 is predicted as a nodulation protein K in H. pylori strain 26695, and a sugar biosynthesis gene in H. pylori strain J99. From the protein sequence comparison, the inventors determined that HP0045 has 37% sequence identity and 57% similarity with both GFSs of E. coli K12 (accession no 8569682) and Y. pseudotuberculosis (accession no. CAB63301). The multiple sequence alignment shows that HP0045 contains many conserved residues, which form characteristic motifs. The conserved Ser-Tyr-Lys catalytic triad in GFS of E. coli is located at S107 and Y136, K140. This triad is involved in catalyzing the reaction (Y136, K140) and interacting with the substrate to stabilize its conformation (S107, K140). Other residues related to NADP(H) binding are also found in HB0045, such as Leu 41, Ala 63. The characteristic GXXGXXG motif is observed at the N-terminus. In addition, GDP-sugar binding sites (Val 180, Leu 184, Trp 202 etc.), phosphate binding sites (Lys 283, Arg209 etc), the 4-keto-sugar interaction sites (Ser107, Ser108, Cys109, Asn165 etc.) can be found in HP0045 (Somers et al., Structure 1998, 6, 1601-6012). This analysis strongly suggests that HP0045 is a GFS gene in the GDP-fucose biosynthesis gene cluster. On the basis of gene similarity, this H. pylori GFS can be classified into the short-chain dehydrogenase/reductase family that catalyzes two distinct reactions at the same active site.

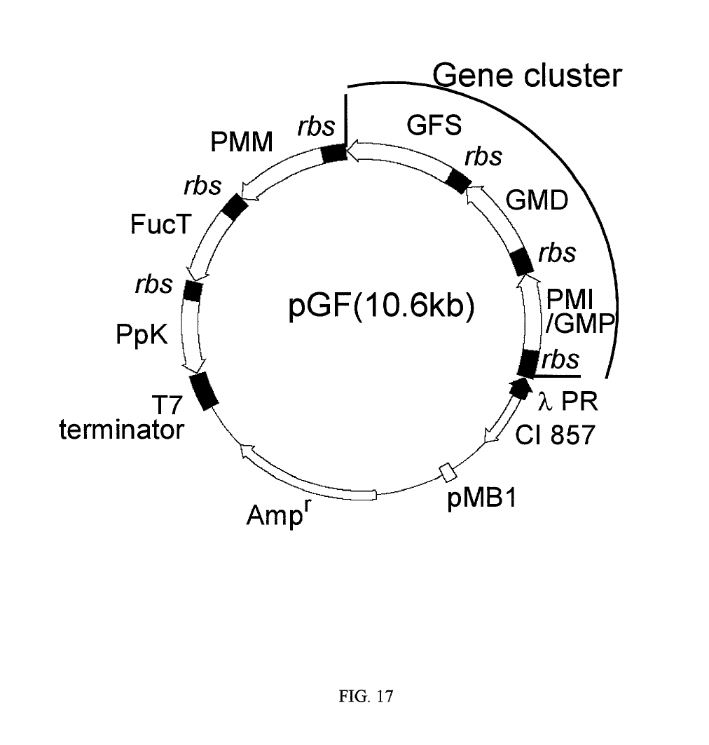

Based on the putative GDP-Fuc gene cluster in H. pylori, the construction of GDP-Fuc regeneration superbug is simplified. This gene cluster (3.5 kb) may be ligated in tandem with another three genes (PMM, PpK and FucT) to construct a recombinant plasmid. The plasmid for the synthesis of fucosylated glycoconjugates using the gene cluster is shown in FIG. 17.

Epimerases

In certain embodiments, it may be useful to use a gene encoding an epimerase. For example, UDP-Gal and UDP-Glc can actually be inter-converted by UDP-galactose 4-epimerase (GaIE) (Wilson and Hogness, J. Biol. Chem. 1964, 239, 2469-2481). Therefore, UDP-Gal and UDP-Glc can be co-regenerated in a single organism through either galactose metabolic pathway (UDP-Gal regeneration) or glucose metabolic pathway (UDP-Glc regeneration). As long as the galE gene coding for GalE is provided heterologously to the organism or overexpressed in the organism, either of these two systems can be used for the regeneration of sugar-nucleotide donor for any glucosyltransferase or galactosyltranferase.

Examples of other epimerases that may be used in conjunction with the present invention include GlcNAc 2-epimerase (GlcNAc; ManNAc), UDP-GlcNAc 2-epimerase (UDP-ManNAc; UDP-GlcNAc), and UDP-GlcNAc 4-epimerase (UDP-GalNAc; UDP-GlcNAc).

Glycosyltransferases

An important aspect of the present invention is the transfer of the sugar moiety from the sugar nucleotide to an acceptor saccharide molecule. This process is carried out by a group of proteins known as glycosyltransferases. Essentially any glycosyltransferase may be used in conjunction with the compositions and methods of the present invention. A great number of glycosyltransferases are known and an extensive list of glycosyltransferases is provided in EP 0870841. A further source of glycosyltransferases, including source organism, EC#, GenBank/GenPept Accession Nos., SwissProt Accession No., and 3D structures, can be found at http://afmb.cnrs-mrs.fr/˜pedro/CAZY/gtf.html (Pedro Coutinho, Glycosoyltransferase Families (last updated Nov. 17, 2000)).

The glycosyltransferase chosen is preferably specific to the sugar nucleotide that is regenerated by the organism. In preferred embodiments, the gene encoding the glycosyltransferase is present in the same plasmid as the nucleotide-regenerating enzymes. In more preferred embodiments, the glycosyltransferase and the nucleotide-regenerating enzymes are co-transcribed and each coding region is preceded by a ribosome binding site.

In preferred embodiments of the present invention, an acceptor is provided to the organism that is capable of being covalently bound to the sugar moiety. Exemplary acceptors include monosaccharides, oligosaccharides, monosaccharides or oligosaccharides linked to a carrier, proteins, peptides, glycoproteins, lipids, glycolipids, glycopeptides, and steroid compounds. Where an acceptor is terminated by a sugar moiety, subsequent sugar moieties will typically be covalently bound to the nonreducing terminus of the terminal saccharide.

Glycosyltransferases typically display specificity in regards to the donor saccharide molecule. Therefore, it is convenient to group them based on their donor specificity.

A. Gal

A large number of glycosyltransferases that transfer galactose (galactosyltransferase) are known. Breton et al. provides an extensive list of prokaryotic and eukaryotic galactosyltransferases and is incorporated herein by reference (J. Biochem. 1998, 123, 1000-1009). Another list can be found at http://stanxterm.aecom.yu.edu/glyc-T/galt.htm (visited Jan. 9, 2001). Galalactosyltransferases include α1,2 galactosyltransferases, such as Gmal2p from yeast (Genbank Acc. No. Z30917), α1,3 galactosyltransferases, such as GGTA1 from mouse (Genbank Acc. No. M26925), β1,4 galactosyltransferases, such as GalT-I from human (Genbank Acc. No. X55415), and ceramide galactosyltransferases, such as CGT from Man (Genbank Acc. No. U30930). Galactosyltransferases that transfer galactose from UDP-Gal to an acceptor molecule include α1,3GalT, β1,4GalT (LgtB), and α1,4GalT (LgtC).

B. Glc

Glycosyltransferases that transfer the glucose to an acceptor molecule are known as glucosyltransferases. Examples of glucosyltransferases include LgtF, Alg5, and DUGT (Heesen et al., (1994) Eur. J. Biochem. 224:71-79; Parker etal., (1995) EMBO J 14:1294-1303).

C. GlcNAc

Glycosyltransferases that transfer the N-acetylglucosamine to an acceptor molecule are known as N-acetylglucosaminyl transferases. A number of N-acetylglucosaminyl transferases are known in the art and include LgtA (β1,3GlcNAc). A list of N-acetylglucosaminyl transferases can be found at http://www.vei.co.uk/TGN/glcnac.htm (lain Wilson (May 24, 1996)) and http://stanxterm.aecom.yu.edu/glyc-T/gnt.htm (visited Nov.21, 2000). N-acetylglucosaminyl transferases include β1,2-N-acetylglucosaminyltransferases, such as MGAT1 from human (Genbank Acc. No. M55621), β1,4-N-acetylglucosaminyltransferases, such as GnT-III from human (Genbank Acc. No. D13789), and β1,6-N-acetylglucosaminyltransferases, such as GnT-V from human (Genbank Acc. No. D17716).

D. GalNAc

Glycosyltransferases that transfer the N-acetylgalactosamine to an acceptor molecule are known as N-acetylgalactosaminyl transferases. A number of N-acetylgalactosaminyl transferases are known and include UDP-GalNAc:2′-fucosylgalactoside-α-3-N-acetylgalactosaminyl transferase. A list of N-acetylgalactosaminyl transferases can be found at http://www.vei.co.uk/TGN/galnac.htm (Iain Wilson (May 24, 1996)). N-acetylgalactosaminyl transferases include α1,3-N-acetylgalactosaminyl transferases (blood group A)(Genbank Acc. No. J05175), β1,3-N-acetylgalactosaminyl transferases (Genbank Acc. Nos. M83651, L25885, U18975, and D17809), CT antigen transferases (Genbank Acc. No. L30104), and polypeptide GalNAc transferases (Genbank Acc. Nos. L17437, X85018, and D85389).

E. GlcA

Glycosyltransferases that transfer glucuronic acid to an acceptor molecule are known as glucuronyltransferases. A list of glucuronyltransferases can be found at http://www.vei.co.uk/TGN/glcuron.htm (lain Wilson (May 24, 1996)). Examples of glucuronyltransferases include UGT1A (Swissprot Acc. No. P22309), UGT1B (Swissprot Acc. No. P36509), UGT1C (Swissprot Acc. No. P35503), UGT1D (Swissprot Acc. No. P22310), and UGT1F (Swissprot Acc. No. P19224). An example of a glucuronyltransferase that recognizes UDP-GlcA to transfer glucuronic acid to an acceptor molecule is UGT2B7.

F. NeuNAc

Sialyltransferases are glycosyltransferases that transfer the N-acetylneuraminic acid to an acceptor. A number of sialyltransferases, including SiaT 0160 (EC 2.4.99.1), are known in the art. (Iain Wilson, http://www.vei.co.uk/TGN/neuac.htm (May 24, 1996)). Sialyltransferases include α2,3-sialyltransferases, such as those desribed by Genbank Acc. Nos. X80503, L13972, X76989, X76988, L23768, X74570, and L23767, α2,6-sialyltransferases, such as those described by Genbank Acc. Nos. X75558, A17362, D16106, X74946, X77775, and L29554, and α2,8-sialyltransferases such as those described by Genbank Acc. Nos. D26360, X84235, U33551, L13445, X80502, and L41680.

Microbial α-2,3-sialyltransferase from N. meningitidis consists of 371 amino acids (Gilbert, M. et al., J. Biol. Chem., 1996, 271, 28271-28276), showing unusual acceptor specificity in that it could use α- and β-terminal Gal residues as acceptors. In addition, (β1,4)-linked and (α1,3)-linked terminal Gal also serve as acceptors. Topology analysis shows that the N-terminal 6 to 24 residues is a non-cleavable signal sequence acting as a membrane anchor, with the catalytic domain facing the periplasmic space. In a preferred embodiment, the non-cleavable signal sequence is replaced by a cleavable signal sequence (pelB leader sequence in pET22b(+) vector, Novagen) so that the sialyltransferase will be exported into periplasmic space with correct folding.

Microbial α2,6SiaT (SiaT 0160, EC 2.4.99.1) has been purified from a marine bacterium Photobacterium damsela (Yamamoto, T. et al., J. Biochem. (Tokyo) 1996, 120, 104- 110). The deduced amino acid sequence does not contain the sialyl binding motif and had no significant similarity to mammalian sialyltransferases. A homologous sequence of SiaT 0160 exists in Pasteurella multocida PM70, with an overall identity of 35% and similarity of 53%. The predicted protein has 412 residues and an N-terminal hydrophobic region that possibly functions as a signal sequence as the one in SiaT 0160. Therefore, the putative protein might be a potential α2,6SiaT. The putative ORF may be cloned, expressed and characterized to determine if it has α2,6SiaT activity.

G. Man

Many mannosyltransferases are known (Iain Wilson, http://www.vei.co.uk/TGN/man.htm (May 24, 1996)). Mannosyltransferases include α1,2-mannosyltransferases such as those described by Genbank Acc. Nos. M81110, X62647, and X89263, α1,3-mannosyltransferases such as that described by Genbank Acc. No. X87947, β1,4-mannosyltransferases such as that described by Genbank Acc. No. J05416, Och1 (Genbank Acc. No. D11095), Mnn1 (Genbank Acc. No. L23753), Mnn10 (Genbank Acc. No. L42540) Dpml (Genbank Acc. No. J04184), and Dol-P-Man:protein mannosyltransferases such as PMT1 (Genbank Acc. No. L19169). Mannosyltransferases that transfer the mannose from GDP-Man to an acceptor saccharide molecule include Alg1 (β1,4-linkage)(Takahashi, T. et al., Glycobiology 2000, 10, 321-327) and Alg2 (α1,3-or α1,6-transferase)(Jackson, B. J. et al., Arch. Biochem. Biophys. 1989, 272, 203-209; Yamazaki, H. et al., Gene 1998, 221, 79-84).

H. Fuc

A list of known fucosyltransferases is provided at http://www.vei.co.uk/TGN/fuc.htm (Iain Wilson, (May 24, 1996)) and http://stanxterm.aecom.yu.edu/glyc-T/fut.html (visited Nov. 21, 2000). Glycosyltransferases that transfer the fucose from GDP-Fuc to an acceptor saccharide molecule include α1,3-FucT (Rizzi, M. et al., Structure 1998, 6, 1453-1465; Martin, S. L. et al., J. Biol. Chem. 1997, 272, 21349-21356), α1,2-FucT (Wang, G. et al., Mol. Microbiol. 1999, 31, 1265-1274), and α1,3/4-FucT (Wang, 1999). Other fucosyltransferases include α1,2-fucosyltransferases, such as those described by Genbank Acc. Nos. m35531, S79196, X91269 and U17894, α1,3/4-fucosyltransferases, such as those described by Genbank Acc. Nos. X87810, X53578, U27326, α1,3-fucosyltransferases, such as those described by Genbank Acc. Nos. M58596, U58860, M81485, L01698, and U08112, and α1,6-fucosyltransferases, such as that described by Genbank Acc. No. D86723.

Vectors

The invention involves engineering an organism to enhance the organism's ability to produce a specific type or class of glycoconjugate. In some instances, the enhancement may comprise increasing the amount of the glycoconjugate the organism could produce naturally. However, in other instances, the organism without engineering is unable to produce the glycoconjugate.

Engineering the organism involves providing one or more heterologous genes to the organism. The heterologous gene may be a gene that is not naturally present in the organism or it may be a gene that is naturally present in the organism but is placed in a different genetic context (e.g., the coding region of the gene is operably linked to a promoter that is not the gene's natural promoter). Typically, the heterologous gene or the resulting protein will have one or more properties differing from the gene in its natural genetic environment.

One method of providing a heterologous gene to an organism is through vectors such as plasmids, phage, phagemids, viruses, artificial chromosomes and the like. The type of vector to be used often will be dependent on the type of organism to be engineered. Preferably, the vector is capable of replicating autonomously within the organism to be engineered. However, the vector also may integrate into the host's genome and replicate along with the rest of the host's genome.

Preferred vectors are expression vectors. Expression vectors contain a promoter that may be operably linked to a coding region. A gene or coding region is operably linked to a promoter when transcription of the gene initiates from the promoter. More than one gene may be operably linked to a single promoter. In preferred embodiments, at least one nucleotide regenerating enzyme gene and at least one glycosyltransferase are both operably linked to the same promoter.

Expression vectors that may be used include, but are not limited to, pUC19 (Gene, 1985, 33, 103), pBluescript II SK+ (Stratagene, La Jolla), the pET system (Novagene; Madison, Wis.), pLDR20 (ATCC 87205), pBTrp2, pBTac1, pBTac2 (Boehringer Mannheim Co.), pKYP10 (Japanese Published Unexamined Patent Application No. 110600/83), pKYP200 (Agric. Biol. Chem., 1984, 48, 669), pLSA1 (Agric. Biol. Chem., 1989, 53, 277), pGEL1 (Proc. Natl. Acad. Sci. USA., 1985, 82, 4306), pSTV28 (manufactured by Takara Shuzo Co., Ltd.), pPA1 (Japanese Published Unexamined Patent Application No. 233798/88), and pCG11 (Japanese Examined Patent Application No. 91827/94). When a yeast strain is used as the host, examples of expression vectors that may be used include YEp13 (ATCC 37115), YEp24 (ATCC 37051), and YCp50 (ATCC 37419).

Essentially any promoter may be used as long as it can be expressed in the engineered organism. A preferred promoter for E. coli is the λ P_{R} promoter. In the presence of the product of the λ C_{I} repressor gene, transcription from the λ P_{R} promoter may be controlled. At temperatures below 37° C., the repressor is bound to the P_{R} promoter and transcription does not occur. At temperatures above 37° C. the repressor is released from the P_{R} promoter and transcription initiates. Thus, by growing the organism containing the vector at 37° C. or above, the genes are expressed.

When the organism is a yeast cell, any promoter expressed in the yeast strain host can be used. Examples include gal 1 promoter, gal 10 promoter, heat shock protein promoter, MF α 1 promoter and CUP 1 promoter.

A ribosome-binding sequence (RBS) (prokaryotes) or an internal ribosome entry site (IRES) (eukaryotes) may be operably linked to the gene. The RBS or IRES is operably linked to the gene when it directs proper translation of the protein encoded by the gene. It is preferred that the RBS or IRES is positioned for optimal translation of the linked coding region (for example, 6 to 18 bases from the initiation codon). In vectors containing more than one gene, it is preferred that each coding region is operably linked to an RBS or IRES. A preferred RBS is AGAAGGAG.

The gene or genes may also be operably linked to a transcription terminator sequence. A preferred terminator sequence is the T7 terminator (pET15b; Novagen 2000 Catalog; Novagen, Madison, Wis.).

The coding region of the gene may be altered prior to insertion into or within the expression vector. These mutants may include deletions, additions, and/or substitutions. When alterations are made, it is preferred that the alteration maintains the desired enzymatic function or specificity of the enzyme. However, in certain embodiments, it may be desired to alter the specificity of the enzyme. For example, one may wish to alter the sugar-nucleotide binding region of the enzyme such that the sugar-nucleotide specificity of the enzyme is changed.

When a heterologous gene is to be introduced into an organism that does not naturally encode the gene, optimal expression of the gene may require alteration of the codons to better match the codon usage of the host organism. The codon usage of different organisms is well known in the art.

The coding region also may be altered to ease the purification or immobilization. An example of such an alteration is the addition of a “tag” to the protein. Examples of tags include FLAG, polyhistidine, biotin, T7, S-protein, and GST (Novagen;pET system). In a preferred embodiment, the gene is altered to contain a hexo-histidine tag in the N-terminus. The protein may be purified by passing through a Ni²⁺ column.

In other embodiments, the coding regions of two or more enzymes are linked to create a fusion protein. In preferred embodiments, an epimerase-glycosyltransferase fusion protein is encoded (Chen et al., J. Biol Chem 2000, 275(41):31594-31600). In a more preferred embodiment, the epimerase-glycosyltransferase fusion protein comprises UDP-galactose 4-epimerase and a 1,3-galactosyltransferase.

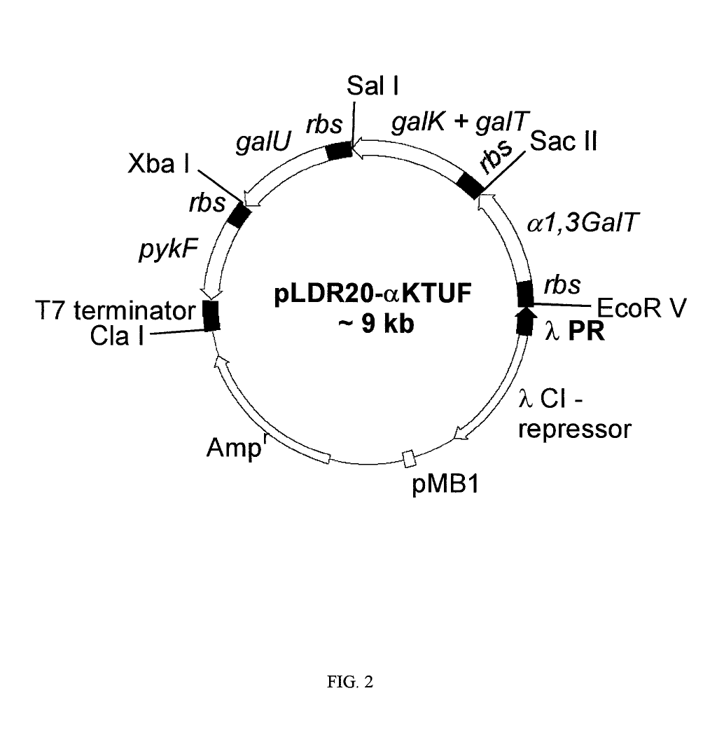

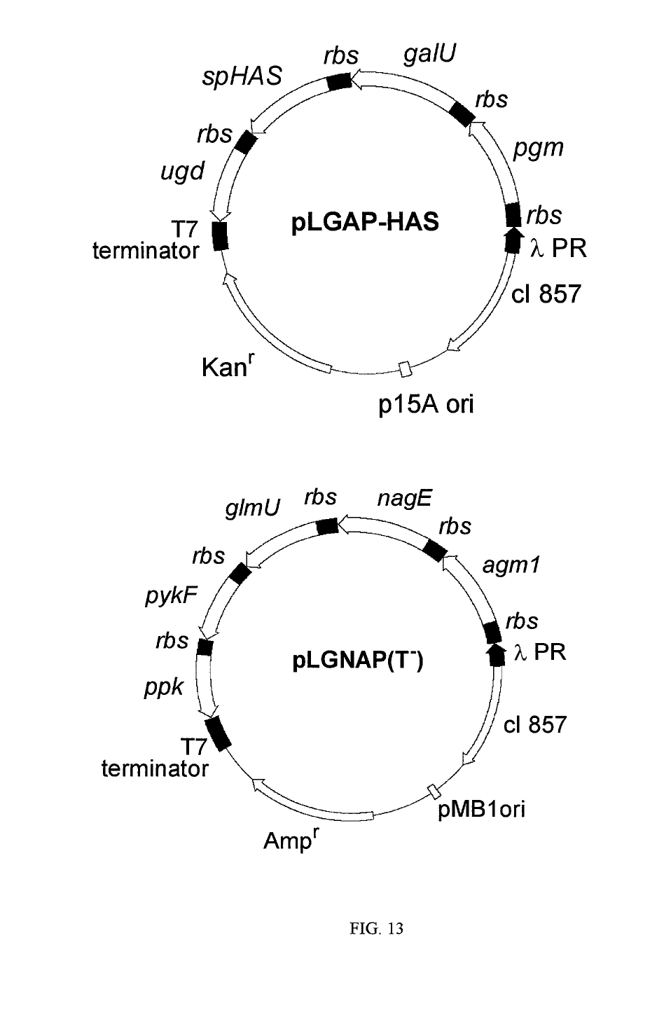

In further preferred embodiments, the expression vector of the present invention comprises at least one gene encoding a sugar-nucleotide regenerating enzyme and at least one glycosyltransferase. The plasmid may also encode one or more enzymes that facilitate the catalysis of a bioenergetic. Preferred plasmids of the present invention include pLDR20-αKTUF (FIG. 2), pLDR20-αKTUN (FIG. 3), pLDR20-αKTUP (FIG. 4), pLDR20-UDPGlc (FIG. 6), pLGNAP (FIG. 7), pLDR20-UDPGalNAc (FIG. 8), pLDR20-GlcA (FIG. 9), pLGAP-HAS (FIG. 13), pLGNAP(T) (FIG. 13), pLDR-Sia (FIG. 10), pL-ManA1A2 (FIG. 11), pL-Mfucα1,3FT (FIG. 12), pLDR20-αES (FIG. 15), and pGF (FIG. 17).

Organisms

A unique aspect of the present invention is the ability to produce large-scale synthesis of glycoconjugates using a single organism. This is accomplished by providing (transfecting) the organism with a vector of the present invention. Essentially any organism may be used as long as it can express the heterologous gene or genes and is capable of producing the desired glycoconjugate when provided with the appropriate bioenergetic and substrates. The organism may be a prokaryote or a eukaryote. Examples of prokaryotes include Esherichia coli BL21 (DE3), Escherichia coli XL1-Blue, Escherichia coli XL2-Blue, Escherichia coli DH1, Escherichia coli MC1000, Escherichia coli KY3276, Escherichia coli W1485, Escherichia coli JM109, Escherichia coli HB101, Escherichia coli No. 49, Escherichia coli W3110, Escherichia coli NY49, Escherichia coli KY8415, Escherichia coli NM522, Bacillus subtilis, Bacillus brevis, Bacillus amyloliquefaciens, Brevibacterium immariophilum ATCC 14068, Brevibacterium saccharolyticum ATCC 14066, Brevibacterium flavum ATCC 14067, Brevibacterium lactofermentum ATCC 13869, Corynbacterium ammoniagenes ATCC 21170, Corynebacterium glutamicus ATCC 13032, Corynbacterium acetoacidophilum ATCC 13870, Microbacterium ammoniaphilum ATCC 15354, Pseudomonas putida, and Serratia marcescens.

The eukaryote may be a yeast, an insect cell, or an animal cell. Examples of yeast include Saccharomyces cerevisiae, Saccharomyces pombe, Candida utilis, Candida parapsilosis, Candida krusei, Candida versatilils, Candida lipolytica, Candida zeylanoides, Candida guilliermondii, Candida albicans, Candida humicola, Pichiafarinosa, Pichia ohmeri, Torulopsis candida, Torulopsis sphaerica, Torulopsis xylinus, Torulopsisfamata, Torulopsis versatilis, Debaryomyces subglobosus, Debaryomyces cantarellii, Debaryomyces globosus, Debaryomyces hansenii, Debaryomyces japonicus, Zygosaccharomyces rouxii, Zygozaccharomyces bailii, Kluyveromyces lactis, Kluyveromyces marxianus, Hansenula anomala, Hansenula jadinii, Brettanomyces lambicus, Brettanomyces anomalus, Schizosaccharomyces pombe, Trichosporon pullulans, and Schwanniomyces alluvius.

Examples of insect cells include SF9 and SF21.

Examples of animal cells include CHO, BHK21, NIH 3T3, 293, and COS.

In a preferred embodiment, the host cell is E. coli, particularly NM522 or DH5α. This organism is well studied and amenable to recombinant technology. Use of this organism in large scale synthesis of compounds is well known in the art. Furthermore, because this organism is LacZ-, it is particularly useful in methods in which lactose is the acceptor molecule. Hydrolization of lactose by LacZ severely decreases the efficiency of the glycoconjugate product in such methods. Generally, if possible, selection of the host organism should take into consideration the existing biochemical and genetic characteristics of the host in order to achieve maximum efficiency.

The inventors also recognize that organisms that naturally express one or more enzymes, or have been engineered to express one or more enzymes, required for a particular glycoconjugate synthesis scheme may be useful. Examples include Escherichia coli which expresses the ceramide glucosyltransferase gene derived from human melanoma cell line SK-Mel-28 (Proc. Natl. Acad. Sci. USA., 1996, 93, 4638), human melanoma cell line WM266-4 which produces β1,3-galactosyltransferase (ATCC CRL 1676), recombinant cell line such as namalwa cell line KJM-1 or the like which contains the β 1,3-galactosyltransferase gene derived from the human melanoma cell line WM266-4 (Japanese Published Unexamined Patent Application No. 181759/94), Escherichia coli (EMBO J., 1990, 9, 3171) or Saccharomyces cerevisiae (Biochem, Biophys. Res. Commun., 1994, 201, 160) which expresses the β 1,4-galactosyltransferase gene derived from human HeLa cells, COS-7 cell line (ATCC CRL 1651) which expresses the rat β 1,6-N-acetylglucosaminyltransferase gene (J. Biol. Chem., 1993, 268, 15381), Sf9 cell line which expresses human N-acetylglucosaminyltransferase gene (J. Biochem., 1995, 118, 568), Escherichia coli which expresses human glucuronosyltransferase (Biochem. Biophys. Res. Commun., 1993, 196, 473), namalwa cell line which expresses human α 1,3-fucosyltransferase (J. Biol. Chem., 1994, 269, 14730), COS-1 cell line which expresses human α 1,3/1,4-fucosyltransferase (Genes Dev., 1990, 4, 1288), COS-1 cell line which expresses human α 1,2-fucosyltransferase (Proc. Natl. Acad. Sci. USA., 1990, 87, 6674), COS-7 cell line which expresses chicken α 2,6-sialyltransferase (Eur. J. Biochem., 1994, 219, 375), COS cell line which expresses human α 2,8-sialyltransferase (Proc. Natl. Acad. Sci. USA., 1994, 91, 7952), Escherichia coli which expresses β 1,3-N-acetylglucosaminyltransferase, β 1,4-galactosyltransferase, β 1,3-N-acetylgalactosaminyltransferase or α 1,4-galactosyltransferase derived from Neisseria (WO 96/10086), Escherichia coli which expresses Neisseria-derived α 2,3-sialyltransferase (J. Biol. Chem., 1996, 271, 28271), Escherichia coli which expresses Heilcobacter pylori-derived α 1,3-fucosyltransferase (J. Biol. Chem., 1997, 272, 21349 and 21357), and Escherichia coli which expresses yeast-derived α 1,2-mannosyltransferase (J. Org. Chem., 1993, 58, 3985). Such organism when further complemented with additional sugar-nucleotide regenerating enzymes will be useful in the methods of the present invention.

Glycoconjugates

In light of the present disclosure, it will become apparent to those of ordinary skill in the art that a great number of different glycoconjugates may be produced by the methods of the present invention if the correct enzymes, precursors, and acceptor molecules are provided to the organism.

Essentially any material may be used as a precursor or acceptor as long as it can be used as a substrate of the glycosyltransferase. The precursor or acceptor may be natural or synthetic. Examples include monosaccharides, oligosaccharides, monosaccharides or oligosaccharides linked to a carrier, proteins, peptides, glycoproteins, lipids, glycolipids, glycopeptides, and steroid compounds. When the glycoconjugate is a glycolipid or a glycoprotein, the glycoconjugate may be O-linked or N-linked.

Specific examples include glucose, galactose, mannose, sialic acid, N-acetylglucosamine, N-acetylgalactosamine, lactose, N-acetyllactosamine, lacto-N-biose, GlcNAc β 1-3Gal β 1-4Glc, GlcNAc β 1-4Gal β 1-4Glc, globotriose, Gal α 1-4Gal β 1-4GlcNAc, 2′-fucosyllactose, 3-fucosyllactose, 3′-sialyllactose, 6′-sialyllactose, 3′-sialyl-N-acetyllactosamine, 6′-sialyl-N-acetyllactosamine, sialyllacto-N-biose, H antigen, Lewis X, Lewis A, lacto-N-tetraose, lacto-N-neotetraose, lactodifucotetraose, 3′-sialyl-3-fucosyllactose, sialyl-Lewis X, sialyl-Lewis A, lacto-N-fucopentaose I, lacto-N-fucopentaose II, lacto-N-fucopentaose III, lacto-N-fucopentaose V, LS-tetrasaccharide a, LS-tetrasaccharide b, LS-tetrasaccharide c, (α 2,3) sialyllacto-N-neotetraose and derivatives thereof, serine, threonine, asparagine and peptides containing these amino acids and derivatives thereof, ceramide and derivatives thereof, saponin and derivatives thereof, and the like. The complex carbohydrate precursor can typically be used at a concentration of from 1 μM to 10 M. Preferably the lower range is 1 mM or 10 mM and the upper range 100 mM or 500 mM.

Examples of the glycoconjugates that may be produced by the methods of the present invention include glycoconjugates containing at least one sugar selected from glucose, galactose, N-acetylglucosamine, N-acetylgalactosamine, glucuronic acid, mannose, N-acetylmannosamine, fucose, sialic acid, lactose, N-acetyllactosamine, lacto-N-biose, GlcNAc β 1-3Gal β 1-4Glc, GlcNAc β 1-4Gal β 1-4Glc, globotriose, Gal α 1-4Gal β 1-4GlcNAc, 2′-fucosyllactose, 3-fucosyllactose, 3′-sialyllactose, 6′-sialyllactose, 3′-sialyl-N-acetyllactosamine, 6′-sialyl-N-acetyllactosamine, sialyllacto-N-biose, A antigen, B antigen, Lewis X, Lewis A, lacto-N-tetraose, lacto-N-neotetraose, lactodifucotetraose, 3′-sialyl-3-fucosyllactose, sialyl-Lewis X, sialyl-Lewis A, lacto-N-fucopentaose I, lacto-N-fucopentaose II, lacto-N-fucopentaose III, lacto-N-fucopentaose V, LS-tetrasaccharide a, LS-tetrasaccharide b, LS-tetrasaccharide c, (α 2,3)sialyllacto-N-neotetraose, lacto-N-difucohexaose I, lacto-N-difucohexaose II, lacto-N-hexaose, lacto-N-neohexaose, disialyllacto-N-tetraose and derivatives thereof, lipopolysaccharide (LPS), such as the LPS of Neisseria meningitidis and Neisseria gonorrhoeae, and complex carbohydrates which contain the just described complex carbohydrates. Specifically, they include complex carbohydrates which contain a sugar having a bond selected from Gal β 1-3Glc, Gal β 1-4Glc, Gal β1-3GlcNAc, Gal β 1-4GlcNAc, Gal β 1-3Gal, Gal β 1-4Gal, Gal β 1-3GalNAc, Gal β 1-4GalNAc, Gal α 1-3Glc, Gal α 1-4Glc, Gal α 1-3GlcNAc, Gal α 1-4GlcNAc, Gal α 1-3Gal, Gal α 1-4Gal, Gal α 1-3GalNAc, Gal α 1-4GalNAc, GlcNAc β 1-3Gal, GlcNAc β 1-4Gal, GlcNAc β 1-6Gal, GlcNAc β 1-3Glc, GlcNAc β 1-4Glc, GlcNAc β 1-3GlcNAc, GlcNAc β 1-4GlcNAc, GlcNAc β 1-6GalNAc, GlcNAc β 1-2Man, GlcNAc β 1-4Man, GlcNAc β 1-6Man, GalNAc β 1-3Gal, GalNAc β 1-4Gal, GalNAc β 1-4GlcNAc, GalNAc α 1-3GalNAc, Man β 1-4GlcNAc, Man α 1-6Man, Man α 1-3Man, Man α 1-2Man, GlcUA β 1-4GlcN, GlcUA β 1-3Gal, GlcUA β 1-3GlcNAc, GlcUA β 1-3GalNAc, NeuAc α 2-3Gal, NeuAc α 2-6Gal, NeuAc α 2-3GlcNAc, NeuAc α 2-6GlcNAc, NeuAc α 2-3GalNAc, NeuAc α 2-6GalNAc, NeuAc α 2-8NeuAc, Fuc α 1-3Glc, Fuc α 1-4Glc, Fuc α 1-3GlcNAc, Fuc α 1-4GlcNAc, Fuc α 1-2Gal and Fuc α 1-6GlcNAc; and complex carbohydrates which contain the just described complex carbohydrates. In this case, the number of sugars contained in the complex carbohydrate containing the sugars may be 10⁴ or below, or 10³ or below.

Methods of Producing Glycoconjugates

The present invention provides for the production of a large variety of glycoconjugates. Generally, the method involves transforming an organism with a vector encoding at least one sugar-nucleotide regenerating enzyme and at least one glycosyltransferase; providing acceptable bioenergetic, precursor, and acceptor molecules to the organism; growing the organism under appropriate conditions to express the enzymes and produce the glycoconjugate; and recovering the glycoconjugate from the organism and/or the growth medium.

Prior to transforming the organism, it may be necessary to construct a vector of the present invention. For the individual cloning of a known or previously unknown gene encoding an enzyme to be used in the compositions and methods of the present invention, the coding region is amplified and isolated, through PCR or essentially any other method of isolating a nucleic acid segment, and cloned into an expression vector. A preferred expression vector is pET15b. The pET15b vector allows for the addition of a N-terminal 6-histidine tag to the protein and a ribosomal binding site to the transcript encoding the protein. The plasmid is then transformed into a host (e.g., BL21 (DE3)) and the protein is expressed. The recombinant protein is purified using a nickel-NTA column and characterized by an enzyme activity assay and enzymatic synthesis (see Example 1).

After it has been determined that the gene encodes a protein with the desired property, in preferred embodiments, the isolated gene, along with the His tag and ribosomal-binding site, encoded in pET is then subcloned into pLDR20. If necessary, the other proteins necessary for sugar-nucleotide regeneration and the glycosyltranserase are cloned into the same vector such that they are co-transcribed.

The organism (preferably a lacZ- strain, such as DH5α or NM522) containing the heterologous genes is then used to produce the glycoconjugate. The organism is provided with an appropriate bioenergetic, along with a substrate and acceptor for the glycosyltransferase. Alternatively, the substrate or acceptor for the glycosyltransferase may be produced by the naturally occurring biochemical pathway or may comprise a molecule that has been produced using a heterologous enzyme provided to the organism. For example, FIG. 1 diagrams a method for producing α1,3Lac. PEP is provided to the organism as a bioenergetic; lactose is provided as an acceptor; and galactose is provided, which is eventually converted to UDP-Gal, a substrate for the glycosyltransferase.

Although monosaccharides and most disaccharides (eg. lactose) and trisaccharides are readily transported into and out of the cells, the transport of oligosaccharides larger than trisaccharide may be problematic in large-scale production. Solutions to this potential problem include: i) permeabilizing reagents can be added to facilitate the transportation of large oligosaccharide acceptors and products; or ii) a secretion mechanism can be applied to help the export of large oligosaccharide products as is the case in hyaluronan synthesis (the hyaluronan was secreted to the media after the production).

After production of the glycoconjugate, the cells may be boiled, pelleted, and the supernatant subsequently run through an ion exchange column. The eluate is then concentrated by evaporation and the concentrated eluate is then run over a gel filtration column. Fractions are collected and tested for the glycoconjugate. The fraction containing the glycoconjugate is then lypholized.

The present invention provides methods of producing glycoconjugates containing glucose. A preferred pathway for producing glucose-containing glycoconjugates is diagrammed in FIG. 6. Also shown in FIG. 6 is a preferred vector for the production of glucose-containing glycoconjugates. In this pathway, polyphosphate is provided as a bioenergetic to an organism containing the vector. Also, glucose is provided, which is converted to UDP-Glc and subsequently added to the acceptor molecule (ROH) to produce GlcOR.

In other embodiments, methods of producing glycoconjugates containing galactose are provided. A preferred pathway for producing galactose-containing glycoconjugates is diagrammed in FIG. 1. Shown in FIG. 2 is a preferred vector for the production of galactose-containing glycoconjugates. In this pathway, polyphosphate is provided as a bioenergetic to an organism containing the vector. Also, galactose is provided, which is converted to UDP-Gal and subsequently added to the acceptor molecule (lactose) to produce GlcOR.

Also provided are methods of producing glycoconjugates containing N-acetylglucosamine. A preferred pathway for producing N-acetylglucosamine-containing glycoconjugates is diagrammed in FIG. 7. Also shown in FIG. 7 is a preferred vector for the production of N-acetylglucosamine-containing glycoconjugates. In this pathway, polyphosphate is provided as a bioenergetic to an organism containing the vector. Also, N-acetylglucosamine is provided, which is converted to UDP-GlcNAc and subsequently added to the acceptor molecule (ROH) to produce GlcNAcOR.

Further provided are methods of producing glycoconjugates containing N-acetylgalactosamine. A preferred pathway for producing N-acetylgalactosamine-containing glycoconjugates is diagrammed in FIG. 8. Also shown in FIG. 8 is a preferred vector for the production of N-acetylgalactosamine-containing glycoconjugates. In this pathway, polyphosphate is provided as a bioenergetic to an organism containing the vector. Also, N-acetylgalactosamine is provided, which is converted to UDP-GalNAc and subsequently added to the acceptor molecule (ROH) to produce GlcNAcOR.

The present invention also provides methods of producing glycoconjugates containing glucuronic acid. A preferred pathway for producing glucuranate conjugates is diagrammed in FIG. 9. Also shown in FIG. 9 is a preferred vector for the production of glucoranate conjugates. In this pathway, polyphosphate is provided as a bioenergetic to an organism containing the vector. Also, glucose is provided, which is converted to UDP-GlcA and subsequently added to the acceptor molecule (ROH) to produce GlcNAcOR by a UDP-glucuronosyltransferase (UGT).

UDP-glucuronosyltransferases (UGTs) are an abundant group of enzymes involved in de-toxification pathways for lipophilic molecules such as phenols, flavones, steroids, bile acids as well as many xenobiotics. In order to synthesize a wide variety of glucuronic acid conjugates, a UDP-GlcA transferase with liberal acceptor specificity is preferred. The significance behind the synthesis of glucuronic acid conjugates is that glucuronidation is not only involved in the detoxification of lipophilic molecules but can also enhance biological activity of a large amount of existing drugs (e.g., morphine-6-O-glucuronide is 50 times more active than morphine). In preferred embodiments, human UDP-GlcA transferase UGT2137 (EC 2.4.1.17) is used. This enzyme has an extremely broad range of substrates. UGT2137 belongs to the 2B subclass of a super-family responsible for glucuronidation of a variety of lipophilic compounds. Its acceptor Kₘ values range from low micromolar to low millimolar. Interestingly, the Kₘ value for UDP-GlcA donor seems dependent on the acceptor. UGT2137 is most active between pH 6.0 and 8.0. Recombinant expression of a human UDP-GlcA transferase in E. coli has been accomplished (Pillot, T. et al., Biochem. Biophys. Res. Commun. 1993, 196, 473-479).

Methods for producing hyaluronan are also provided. Hyaluronan (or hyaluronic acid), a co-polymer of glucuronic acid and N-acetyiglucosamine, is common in the extracellular spaces of multicellular organisms where it forms a viscous, compression resistant matrix. In prokaryotes, hyaluronate is found in the anti-phagocytic capsule formed by virulent species such as Streptococcus pyogenes and Streptococcus pneumoniae, where it helps the bacterium evade the host immune system.

Hyaluronan synthases (HAS) is the first sugar transferase shown to have the ability to utilize two different UDP-sugar donors. HAS enzymes are membrane proteins that require divalent metal ion (Mg²⁺ or Mn²⁺) for optimal activity, and show two- to five-fold higher apparent affinity for the UDP-GlcA substrate than for UDP-GlcNAc. For hyaluronic acid synthesis, it is preferred to use the spHas gene encoding Hyaluronan synthase (spHAS) from Streptococcus pyogenes. This gene encodes a 45 kDa protein with 395 residues and was first cloned and identified in 1993 and that was later shown to be expressed in the membrane fraction (DeAngelis, P. L. et al., J. Biol. Chem. 1993, 268, 19181-19184; Tlapak-Simmons, V. L. et al., J. Biol Chem. 1999, 274, 4239-4245). spHAS activity is dependent on lipids. Maximal activity is obtained in the presence of bovine cardiolipin being about twice the activity when E. coli cardiolipin is used. The enzyme exhibits Kₘ values of 40±4 μM for UDP-GlcA and 149±3 μM for UDP-GlcNAc.

To produce large-scale synthesis of HA inexpensively, a preferred method regenerates both UDP-GlcNAc and UDP-GlcA, as well as expresses a hyaluronan sythase. This may be accomplished by engineering an organism to over-express all the enzymes necessary for the precursor generation by means of a dual plasmid system (FIG. 13) comprising plasmid pLGNAP(T-) and plasmid pLGAP-HAS. In preferred embodiments, these two plasmids are constructed with compatible origins of replication to be able to coexist in the same organism. They also contain different antibiotic resistance genes for easy selection of recombinant strains containing both plasmids. Both plasmids contain the X promoter region that is under the control of the temperature sensitive λ cI857 repressor. This enables simultaneous expression of proteins from both plasmids once the incubation temperature is raised.

Omitting the gene of GlcNAc transferase, pLGNAP(T-) encodes all of the enzymes for the regeneration of UDP-GlcNAc (FIG. 7).

Plasmid pLGAP-HAS contains enzymes for the production of UDP-GlcA as well as the hyaluronan synthase. Since ppK gene is incorporated in the plasmid pLGNAP(T⁻), no addition copy of ppK is included in pLGAP-HAS for the synthesis of hyaluronan. This plasmid has a kanamycin resistance gene and p15A replication origin compatible for the pMB 1 origin in plasmid pLGNAP(T⁻).

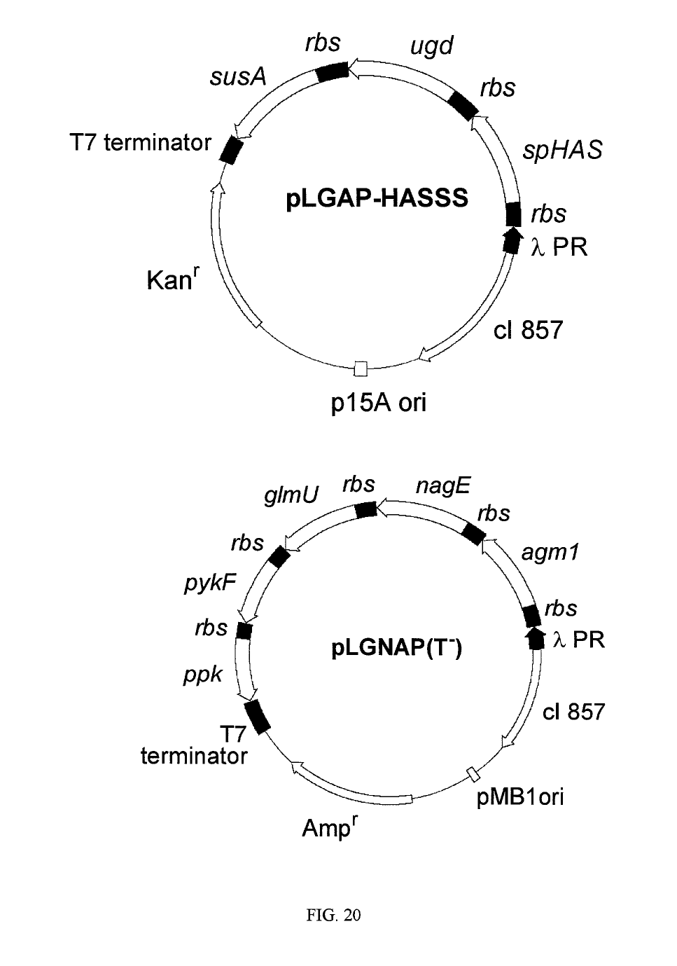

Plasmids useful in methods of synthesis of hyaluronan wherein sucrose is used are shown in FIG. 20.

It is also important to mention that HA is secreted out of the cell as it is synthesized. This greatly facilitates the purification effort and will exclude the need to permeabilize the cells. In embodiments wherein the glycoconjugate is secreted from the cells, the growth medium may be continuously or intermittently removed from the cells, the glycoconjugate is isolated from the removed medium, and the medium is subsequently returned to the cells or fresh medium is added to the cells. A method for continuously obtaining cellulose secreted from bacteria is disclosed in U.S. Pat. No. 6,132,998. In light of the present invention, one of ordinary skill in the art would understand how to modify the method of U.S. Pat. No. 6,132,998 for the production of glycoconjugates in accordance with the present invention.

Other important glycoconjugates that may be produced by the methods of the present invention are sialic acid-containing glycoconjugates. Sialic acids (N-acetylneuraminic acid, NeuNAc) exist as the terminal saccharides in a variety of glycoproteins and glycolipids on the mammalian cell surface as well as on some neuroinvasive bacteria such as Neisseria meningitis Group B and E. coli K1. Sialic acids containing structures play important roles in cell-cell recognition. Therefore, the synthesis of sialylated conjugates is of great importance in developing novel carbohydrate-based therapeutic agents (Fryer and Hockfield, Curr. Opin. Neurobiol. 1996, 6, 113-118; Rougon, G. Eur. J. Cell Biol. 1993, 61, 197-207; Phillips, G. R. et al., Brain Res. Dev. Brain Res. 1997, 102, 143-155; Liu, T. Y. et al.; J. Biol. Chem. 1971, 246, 4703-4712; Egan, W. et al., Biochemistry 1977, 16, 3687-3692).

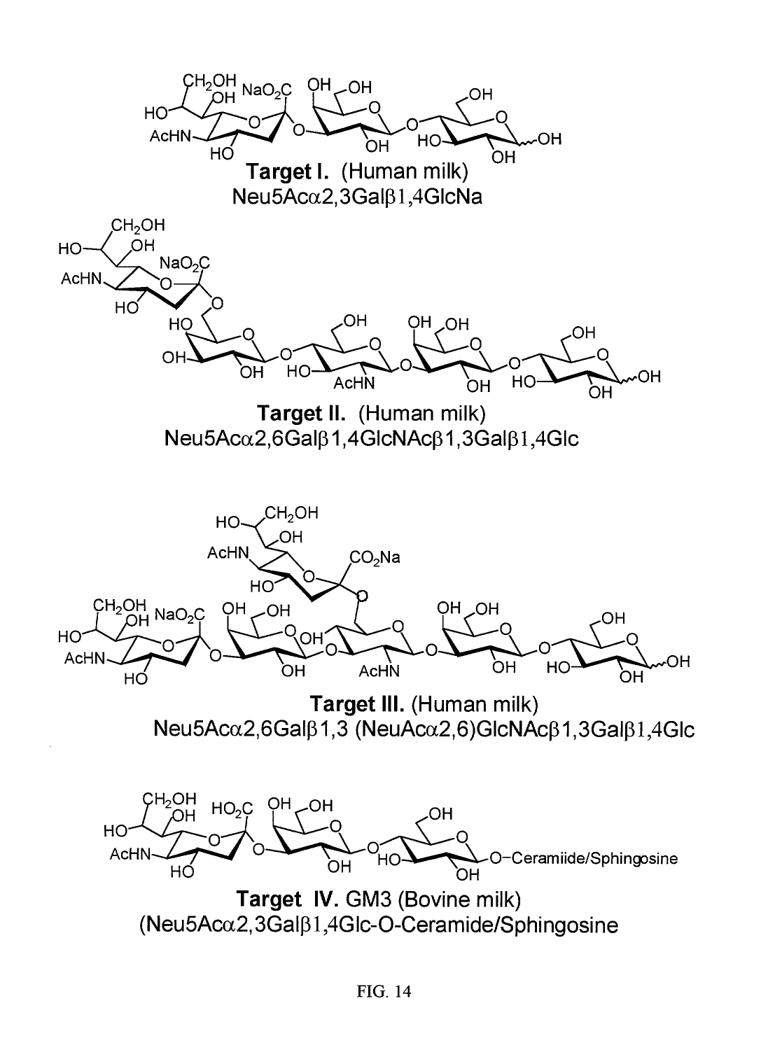

The biosynthesis of sialylated glycoconjugate typically requires CMP-NeuNAc synthesized from CTP and sialic acid. In a preferred embodiment, nanA (sialic acid aldolase), neuA (CMP-NeuNAc synthetase), cmk (CMP kinase), and ppk (polyphosphate kinase) from E. coli, along with the gene for α2,3 (or α2,6)-sialyltransferase (SiaT), are cloned into one plasmid (FIG. 10). Four exemplary glycoconjugates that may be produced by the methods of the present invention are shown in FIG. 14.

Also provided by the present invention are methods of producing mannose-containing glycoconjugates. A preferred pathway for producing mannose-containing glycoconjugates is diagrammed in FIG. 11. Also shown in FIG. 11 is a preferred vector for the production of mannose-containing glycoconjugates (pL-ManA1A2). In this pathway, polyphosphate is provided as a bioenergetic to an organism containing the vector. Also, mannose is provided, which is converted to GDP-Man and subsequently added to the acceptor molecule (ROH) to produce ManOR.

Further provided are methods of producing fucose-containing glycoconjugates. A preferred pathway for producing fucose-containing glycoconjugates is diagrammed in FIG. 12. Also shown in FIG. 12 is a preferred vector for the production of fucose-containing glycoconjugates (pL-Mfucα1,3FT). In this pathway, polyphosphate is provided as a bioenergetic to an organism containing the vector. Also, mannose is provided, which is converted to GDP-Fuc and subsequently added to the acceptor molecule (ROH) to produce FucOR.

Conditions for Producing Glycoconjugates

The methods of the present invention are adaptable to small scale and large scale (fermentors) production of glycoconjugates. Culturing of the organisms for use in the present invention may be carried out in accordance with the usual culturing process.

For example, where the organism is a microorganism, such as E. coli, the medium for use in the culturing of the microorganism may be either a nutrient medium or a synthetic medium, so long as it contains carbon sources, nitrogen sources, inorganic salts and the like, which can be assimilated by the microorganism and it can perform culturing of the microorganism efficiently.

Examples of the carbon sources include those which can be assimilated by the microorganism, such as carbohydrates (for example, glucose, fructose, sucrose, lactose, maltose, mannitol, sorbitol, molasses, starch, starch hydrolysate, etc.), organic acids (for example, pyruvic acid, lactic acid, citric acid, fumaric acid, etc.), various amino acids (for example, glutamic acid, methionine, lysine, etc.), and alcohols (for example, ethanol, propanol, glycerol, etc.). Also useful are natural organic nutrient sources, such as rice bran, cassava, bagasse, corn steep liquor, and the like.

Examples of the nitrogen sources include various inorganic and organic ammonium salts (for example, ammonia, ammonium chloride, ammonium sulfate, ammonium carbonate, ammonium acetate, ammonium phosphate, etc.), amino acids (for example, glutamic acid, glutamine, methionine, etc.), peptone, NZ amine, corn steep liquor, meat extract, yeast extract, malt extract, casein hydrolysate, soybean meal, fish meal or a hydrolysate thereof and the like.

Examples of the inorganic substances include potassium dihydrogen phosphate, dipotassium hydrogen phosphate, sodium dihydrogen phosphate, disodium hydrogen phosphate, magnesium phosphate, magnesium sulfate, magnesium chloride, sodium chloride, calcium chloride, ferrous sulfate, manganese sulfate, copper sulfate, zinc sulfate, calcium carbonate, and the like. Vitamins, amino acids, nucleic acids and the like may be added as occasion demands.

The culturing is carried out under aerobic conditions by shaking culture, aeration stirring culture or the like means. The culturing temperature is preferably from 15 to 45° C., and the culturing time is generally from 5 to 96 hours. The pH of the medium is maintained at 3.0 to 9.0 during the culturing. Adjustment of the medium pH may be carried out using an inorganic or organic acid, an alkali solution, urea, calcium carbonate, ammonia and the like. Also, antibiotics (for example, ampicillin, tetracycline, etc. ) may be added to the medium during the culturing as occasion demands.

In some embodiments, a microorganism transformed with an expression vector in which an inducible promoter is used. Culturing may be adjusted such that induction of the promoter is regulated (e.g., adjustment of culturing temperature). Alternatively, where a promoter is induced by a compound, an inducer may be added to the medium as occasion demands. For example, isopropyl-β-D-thiogalactopyranoside (IPTG) or the like may be added to the medium when a microorganism transformed with an expression vector containing lac promoter is cultured, or indoleacrylic acid (IAA) or the like may by added when a microorganism transformed with an expression vector containing trp promoter is cultured.

When animal cells are used for producing the complex carbohydrate of the present invention, the preferred culture medium is generally RPMI 1640 medium, Eagle's MEM medium or a medium thereof modified by further adding fetal calf serum, and the like. The culturing is carried out under certain conditions, for example, in the presence of 5% CO₂. The culturing is carried out at a temperature of preferably from 20 to 40° C. for a period of generally from 3 to 14 days. As occasion demands, antibiotics may be added to the medium.

When insect cells are used for producing glycoconjugates of the present invention, culturing of the insect cells can be carried out in accordance with known processes (e.g., J. Biol. Chem., 1993, 268, 12609).

Kits

Further, provided for by the present invention are kits containing one or more compositions of the present invention for the production of glycoconjugates. The kit may include a plasmid encoding at least one nucleotide-regenerating enzyme and at least one glycosyltransferase. A kit of the present invention may comprise an organism. The organism may have been transfected with a plasmid of the present invention or the plasmid may be included in the kit with the organism. Furthermore, the bioenergetic that the organism has been engineered to utilize to produce a glycoconjugate also may be included in the kit.

### EXAMPLES

The following examples are included to demonstrate embodiments of the invention. It should be appreciated by those of skill in the art that the techniques disclosed in the examples that follow represent techniques discovered by the inventors to function well in the practice of the invention, and thus can be considered to constitute preferred modes for its practice. However, skilled in the art should, in light of the present disclosure, appreciate that many changes can be made in the specific embodiments that are disclosed and still obtain like or similar results without departing from the spirit and scope of the invention.

### Example 1

### Production of α-Gal Epitopes by Recombinant E. coli Using PykF

This example describes the construction of a metabolic pathway-engineered E. coli strain, referred to as a superbug, harboring all the enzymes in the biosynthetic pathway of α-Gal epitopes. Also, described is the use of the superbug to produce oligosaccharides with a terminal Galα1,3Gal sequence.

A. Materials and Methods Used in This Example

1. Bacterial Strains and Plasmids

Plasmid vector pLDR20 (ATCC catalog no. 87205) and E. coli K-12, substrain MG1655 (ATCC catalog no. 47076) were purchased from American Tissue Culture Collection. Plasmid vector pET15b and E. coli competent cell BL21(DE3) [F ompT hsdS_{B}(r_{B}⁻m_{B}⁻) gal dcm (DE3)] were from Novagen Inc., Madison, Wis.. Plasmid pET15b-α1,3GalT was constructed as described in Fang et al. (J. Am. Chem. Soc. 1998, 120, 6635-6638). E. coli competent cell DH5α (lacZΔM15 hsdR recA) was from Gibco-BRL Life Technology, Rockville, Md. Competent cell NM522 {supE thi-1 Δ(lac-proAB) Δ(mcrB-hsdSM)5(r_{K}⁻ m_{K}⁺) [F'proABlacl^{q}ZΔM15]} was from Strategene, La Jolla, Calif.

2. Cloning, Overexpression and Purification of Individual Enzymes.

DNA manipulations were performed as described in Fang et al. (1998).

The galK gene (primers 5′-GATCATATGAGTCTGAAAGAAAAAACAC-3′ (SEQ ID NO: 2) and 5′-CGCGGATCCTCAGCACTGTCCTGCTCCTTG-3′ (SEQ ID NO: 3)), galT gene (primers 5′- GGATCCATATGACTAGTATGACGCAATTTAATCCC-3′ (SEQ ID NO: 4) and 5′-AGCGGATCCTTACACTCCGGATTCGCG-3′ (SEQ ID NO: 5)),

galU gene (primers 5′-GGATCCTCGAGATGGCTGCCATTAATACG-3′ (SEQ ID NO: 6) and 5′-CGCGGATCCACTAGTTTACTTCTTAATGCCCATCTC-3′ (SEQ ID NO: 7)), and

pykF gene

(primers 5′-GGATCCATATGAAAAAGACCAAAATTGTTTGCACC-3′ (SEQ ID NO: 8) and 5′-CGCGGATCCACTAGTTTACAGGACGTGAACAGATGC-3′ (SEQ ID NO: 9)) were cloned from E. coli K-12 and inserted into NdeI and BamHI (XhoI and BamHI for galU) restriction sites of pET15b vector, respectively. The resulting plasmids pET15b-galK, pET15b-galT, pET15b-galU, and pET15b-pykF were transformed into E. coli cloning host strain DH5α and then expression host strain BL21 (DE3), respectively. Selected clones were characterized by restriction mapping. The expression and purification of individual enzymes from the cell lysate were as described in Fang et al. (1998).

Briefly, the overexpression of the enzymes were induced by 0.4 mM IPTG (isopropyl-1-thio-β-D-galactospyranoside) for 3 hr at 37° C. in a C25 incubator shaker (New Brunswick Scientific Co., Inc., Edison, N.J.). The cell lysate and inclusion bodies were separated by centrifugation at 12,000 rpm for 20 min. From cell lysate, the active enzymes were purified using a Ni²⁺-NTA (nickel-nitriloacetic acid) agarose affinity column which binds to the N-terminal His₆-tag sequence in the recombinant proteins. After elution, the fractions containing the purified enzyme (detected by a UV-Vis spectrometry) were combined and dialyzed for enzyme activity assays and enzymatic reactions.

3. Enzymatic Activity Assay for Galactokinase.

The activity assays for GalK were performed at room temperature (24° C.) for 30 min in a final volume of 100 μl in HEPES buffer (100 mM, pH 7.4) containing α-D-[6-³H]galactose (0.5 mM, final specific activity of 1000 cpm/nmole) and ATP (50 mM). 5 ATP was omitted for the blank. The reaction was stopped by adding 0.8 ml of Dowex 1×8-200 chloride anion exchange resin suspended in water [resin:H₂O (vol/vol)=1:1]. After centrifugation, supernatant (0.4 ml) was collected in a 20 mil plastic vial and ScintiVerse BD (5 ml) was added. The vial was vortexed thoroughly before the radioactivity of the mixture was counted in a liquid scintillation counter (Beckmann LS-3801 counter). One unit of enzyme activity is defined as the amount of enzyme that produces 1 μmole of galactose-1-phosphate per minute at 24° C.

4. Enzymatic Activity Assay for Galactose-1-Phosphate Uridylyltransferase

This was a two-step assay. In the first step, GalT catalyzed reactions were performed at room temperature (24° C.) for 15 min in a final volume of 250 μl HEPES buffer (100 mM, pH 7.4) containing 1.6 mM Gal-1-P, 2.8 mM UDP-glucose, and 100 μl of enzyme solution. A blank was performed with water replacing Gal-1-P. The reaction was stopped by adding cold NaCl solution (0.5 ml, 0.15 M) and immediately transferring the tube to a boiling water bath for 5 min to terminate the reaction. The contents of the tubes were cooled to room temperature and vortexed vigorously to break up the coagulum. After centrifugation at 1400×g for 15 min, the clear supernatant (0.2 ml) was subjected to the UDP-glucose assay in a cuvette with a total volume of 1 ml containing 0.03 M Tris-acetate buffer, pH 8.7, 1.36 mM NAD, 0.2 ml sample (supernatant from the previous procedure) and 3.2 mU UDP-glucose dehydrogenase. The OD change at 340 nm was monitored by a UV spectrophotometer (HP 8453 Spectrophotometer, Hewlett-Packard Com.). One unit of enzyme activity was defined as the amount of enzyme that produces 1 μmole of UDP-galactose per minute at 24° C.

5. Enzymatic Activity Assay for Glucose-1-Phosphate Uridylyltransferase

A two-step assay was carried out to detect the GalU activity. In the first step, GalU catalyzed reactions were performed at room temperature (24° C.) for 15 min in a final volume of 250 μl containing 1.6 mM Glc-1-P, 2.8 mM UTP, 10 mM MgCl₂ and 100 μl enzyme solution. A blank was performed with water replacing Glc-1-P. The reaction was stopped by adding cold NaCl solution (0.5 ml, 0.15 M) and immediately transferring the tube to a boiling water bath for 5 min to terminate the reaction. The contents of the tubes were cooled to room temperature and vortexed vigorously to break up the coagulum. After centrifuge at 1400×g for 15 min, the clear supernatant (0.2 ml) was subjected to the UDP-glucose assay in a cuvette with a total volume of 1 ml containing 0.03 M Tris-acetate buffer, pH 8.7, 1.36 mM NAD, 0.2 ml sample (supernatant from the previous procedure) and 3.2 mU UDP-glucose dehydrogenase. The OD change at 340 nm was monitored by a UV spectrophotometer (HP 8453 Spectrophotometer, Hewlett-Packard Com.). One unit of enzyme activity was defined as the amount of enzyme that produces 1 μmole of UDP-glucose per minute at 24° C.

6. Enzymatic Activity Assay for Pyruvate Kinase

In a 10 mm light path cuvette was pipette successively with a total volume of 1 ml solution containing 0.1 M Tris-HCl buffer, pH 8.0, 0.5 mM EDTA, 0.1 M KCl, 10 mM MgCl₂, 0.2 mM NADH, 1.5 mM ADP, 60 mU lactate dehydrogenase, and 5 mM PEP. A blank assay was carried out with water replacing ADP. The reactions were performed at room temperature (24° C.) and the absorbance at 340 nm was monitored by a UV-spectrophotometer. One unit of enzyme activity was defined as the amount of enzyme that produces 1 μmole of pyruvate per minute at 24° C.

7. Stepwise Radioactivity Assays

Three steps of radioactivity assay were carried out using the combination of purified enzymes. Radio-labeled galactose was used. The first step assay was to test the combined activity of GalK, GalT and α1,3GalT. The enzyme assay was performed at 37° C. for 2 h in a final volume of 100 μl containing HEPES buffer (100 mM, pH 7.4), MnCl₂ (10 mM), D-[6-³H]galactose (0.5 mM, 20,000 dpm), ATP (5 mM), UDP-Glc (5 mM), Lac-grease (0.14 mM), and enzyme solutions (20 μl of GalK, GalT and α1,3GalT respectively). ATP was omitted for the blank. The reaction was stopped by adding 0.5 ml of ice cold water.

The mixture was then passed through a Sep-Pak C₁₈ cartridge pre-washed with MeOH (20 ml) and H₂O (20 ml). The cartridge was then washed with 30 ml of water before the radio-labeled product (Galα1,3Lac-grease) was eluted with MeOH (3.5 ml). The eluate was collected in a 20 ml plastic vial and ScintiVerse BD (10 ml) was added. The vial was vortexed thoroughly before the radioactivity of the mixture was counted in a liquid scintillation counter (Beckmann LS-3801 counter).

The second step assay was to test the combined activities of GalK, GalT, α1,3GalT and GalU. The procedures were the same as the first step assay except that the reaction mixture consisted of HEPES buffer (100 mM, pH 7.4), MnCl₂ (10 mM), D-[6-³H]galactose (0.5 mM), ATP (5 mM), UTP (5 mM), Glc-1-phosphate (0.5 mM), Lac-grease (0.14 mM), and enzyme solutions of GalK, GalT, a1,3GalT, and GalU (20 μl respectively). The third step assay was the whole cycle assay, the reaction mixture consisted of HEPES buffer (10 mM, pH 7.4), MnCl₂ (10 mM), MgCl₂ (10 mM), KCl (100 mM), D-[6-³H]galactose (0.5 mM), ATP (5 mM), 5 mM PEP, 0.5 mM Glc-1-phosphate, UDP (0.5 mM), Lac-grease (0.14 mM), and enzyme solutions of all of the five enzymes (20 μl of GalK, GalT, α1,3GalT, GalU, and PykF respectively) with a final volume of 150 μl.

8. Stepwise Synthesis of α-Gal

The principle of the superbug was further confirmed by the synthesis of α-Gal on preparative (100 mg) scales using the combination of purified enzymes. Five steps of synthesis were carried out under similar conditions with stepwise-addition of enzymes and varying starting materials. Generally, purified enzyme/enzymes (0.5 ml for each) was/were added to the reaction mixture to a total of 5 ml volume. The reactions were carried out at room temperature for 3 days and then stopped by incubating in boiling water for 10 min to precipitate the enzymes. After centrifigation for 20 min, the supernatant consequently was passed through anion exchange resins and cation exchange resins, concentrated and loaded to a G-15 gel filtration column (120×4 cm) with water as the eluent. The desired fractions were collected and lyophilized to yield the product.

For the first step reaction, purified α1,3 GalT was added to a HEPES buffer (100 mM, pH=7.4) containing UDP-galactose (153 mg), MnCl₂ (10 mM), and lactose (86 mg). For the second step reaction, two enzymes (GalT and α1,3GalT) were included, and the starting materials were Gal-1-P (84 mg), UDP-Glc (153 mg), lactose (86 mg) and MnCl₂ (10 mM). In the third step reaction, three enzymes (GalK, GalT and α1,3GalT) were included, and the starting materials were ATP (138 mg), Gal (45 mg), UDP-Glc (153 mg), lactose (86 mg) and MnCl₂ (10 mM). For the fourth step reaction, four enzymes (GalU, GalK, GalT and α1,3GalT) were included, and the starting materials were ATP (14 mg), Gal (5 mg), UTP (12 mg), Glc-1-P (1 mg), lactose (9 mg), MgCl₂ (10 mM), and MnCl₂ (10 mM). For the fifth step reaction, five enzymes (PykF, GalU, GalK, GalT and α1,3GalT) were included, and the starting materials were ATP (14 mg), Gal (5 mg), PEP (5 mg), Glc-1-P (1 mg), UDP (2 mg), lactose (9 mg), MgCl₂ (10 nM), KCl (100 mM), and MnCl₂ (10 mM).

9. Construction of the Pathway-Engineered Organism (Superbug)

The plasmid for the α-Gal superbug was constructed by insertion of five genes into the pLDR20 plasmid vector. Firstly, galU gene was inserted, primers GalU-N

(5′-CCGGATATCCCGCGGGTCGACAATAATTTTGTTTAACTTTAAGAAGG-3′ (SEQ ID NO: 10)) and GalU-C

(5′- GCATCGATGGTCTAGAGGATCCTTACTTCTTAATGCCCATCTC-3′ (SEQ ID NO: 11)) were used in the PCR to introduce EcoRV, SacII, SalI (by GalU-N), and XbaI, ClaI (by GalU-C) restriction sites, respectively. The template DNA was plasmid pET15b-galU. The PCR product including galU gene, codons for N-terminal His₆-tag and ribosomal binding site was digested with EcoRV and ClaI and inserted into the multiple-cloning sites of the pLDR20 vector previously cut with the same restriction enzymes. The positive clones harboring plasmid pLDR20-U were confirmed by restriction mapping and the expression of the GalU was confirmed by SDS-PAGE.

Secondly, primers α1,3GalT-N

(5′-GGATCCATATGACTAGTGATATCAATAATTTTGTTTAACTTTAAGAAGG-3′ (SEQ ID NO: 12)) and α1,3GalT-C

(5′- CCATCGATGTCGACCCGCGGTCAGACATTATTTCTAACCAC-3′ (SEQ ID NO: 13)) were used to amplify the α1,3GalT gene with ribosomal binding site and codons for His₆-tag from pre-constructed plasmid pET15b-α1,3GalT. The PCR product was digested and inserted into EcoRV and SacII two restriction sites of the plasmid pLDR20-U to form plasmid pLDR20-αU.

Thirdly, the smaller fragment of the pET15b-PykF digestion products (digested with XbaI and ClaI), containing a pykF gene with ribosomal binding site and a T7 terminator, was purified and inserted into XbaI and ClaI two restriction sites of the plasmid pLDR20-αU to form plasmid pLDR20-αUF. Lastly, galK and galT genes were amplified from plasmid pET15b-galKT (constructed by inserting the gene sequence encoding both galK and galT genes into Nde I and BamH I two restriction sites of pET 15b vector) using primers GalKT-N

(5′- TCCCCGCGGCCCGGGAATAATTTTGTTTAACTTTAAGAAGG-3′ (SEQ ID NO: 14)) and GalKT-C

(5′- CGCGTCGACTCAGCACTGTCCTGCTCCTTG-3′ (SEQ ID NO: 15)). The PCR product was digested and inserted into Sac II and Sal I two restriction sites of plasmid pLDR20-αUF to form plasmid pLDR20-αKTUF. This final plasmid harboring five genes was transformed into DH5α and NM522 competent cells.

10. Substrates Analysis for the Synthesis of α-Gal with Superbug

The expression of the target genes in the superbug was initiated by increasing the temperature of bacterial culture from 30° C. to 40° C. After the expression continued for 3-3.5 hr, the cells were separated by centrifugation (4,000 g×20 min), suspended in 25 ml/l bacterial culture of Tris-HCl buffer (20 mM, pH 8.5) containing 1% Triton X-100, and ready for the synthesis. Before large-scale synthesis was carried out, small-scale syntheses of α-Gal were performed with omitting of one or more substrates required in cell-free synthesis. In a total of 1 ml volume in a microcentrifuge tube, 100 μl of metal ion mixture containing KCl (1 M), MgCl₂ (100 mM), MnCl₂ (100 mM) in a HEPES buffer (1 M, pH 7.4) and 50 μl of each of the following stock solutions: Gal (200 mM), Glc (200 mM), ATP (100 mM), LacOH (100 mM) (or LacOBn), PEP (100 mM), UDP (10 mM), Glc-1-P (10 mM), bacteria solution (0.55 ml) was added. Reaction conditions were varied by substituting one or more of the substrates with a same volume of water. After the reactions were carried out for 24 hr at room temperature in a rotor, the formation of the products were characterized by TLC [i-PrOH:H₂O:NH₄OH=7:3:2 (vol/vol/vol)] and the yields were quantified by high performance liquid chromatography [HPLC, MICROSORB™-100Å amino column, mobile phase: CH₃CN:H₂O=65:35 (vol/vol) (CH₃CN:H₂O=80:20 for LacOBn as acceptor(vol/vol))].

11. Synthesis of α-Gal with the Superbug

Gram-scale synthesis of α-Gal was carried out using LacOH as acceptor in a total of 500 ml volume in a flask (1 l) at room temperature with agitation (700 rpm) by a magnetic stirrer. Briefly, to LacOH (4.28 g, 12.5 mmol), Gal (4.50 g, 25 mmol), Glc (4.50 g, 25 mmol), ATP (0.55 g, 1.0 mmol), UDP-Glc (0.61 g, 1.0 mmol), Glc-1-P (0.31 g, 1.0 mmol) in a 1 liter flask was added 50 ml of each of the following stock solution: HEPES buffer (0.5 M, pH 7.4), MnCl₂ (0.1 M), MgCl₂ (0.1 M), and KCI (1 M). Then superbug cells [50 g in 300 ml Tris-HCl buffer (20 mM, pH 8.5) containing 1% Triton X-100, obtained from 10 l bacterial culture] were added to bring the total reaction mixture volume to 500 ml. The reaction was monitored by TLC [i-PrOH:H₂O: NH₄OH=7:3:2 (vol/vol/vol)] and high performance liquid chromatography (HPLC). After 36 hr, when TLC analysis indicated that reaction was complete, the reaction was stopped by putting the flask in boiling water for 10 min. The pellet was removed by centrifugation at 5,000×g for 20 min and washed twice with 50 ml deionized water. The combined supernatants consequently were passed through an anion exchange column and a cation exchange column and concentrated. Part of the elute (1/10 volume) was loaded to a Sephadex G-15 gel filtration column (120 cm×4 cm) with water as the mobile phase. The desired fractions were pooled and lyophilized to give Galα1,3LacOH (0.41 gram).

12. Analysis of Oligosaccharide Products

¹H and ¹³C NMR (400 MHz) spectra were obtained using a 400-MHz Varian VXR400 NMR or a 500-MHz Varian NMR spectrometer with the chemical shift expressed as parts per million downfiled using deutrated water as solvent. Thin-layer chromatography was conducted on Baker Si250F silica gel TLC plates with a fluorescent indicator. The following data were obtained:

Galα1,3Galβ1,4GlcOH

¹NMR (D₂O): δ4.33 (d, J=7.5 Hz, 1H), 4.48 (d, J=8.0 Hz), 4.96 (d, J=4.0 Hz, 1H), 5.04 (d, J=3.5 Hz). Selected anomeric ¹³C NMR (D₂O): δ91.69, 95.28, 95.64, 102.70; MS (FAB) 527 (M+Na⁺).

Galα1,3Galβ1,4GlcOBn

¹H NMR (D₂O): δ7.33-7.27 (m, 5 H), 4.99 (d, J=3.5 Hz, 1 H), 4.79 (d, J=11.5 Hz, 1 H), 4.61 (d, J=11.5 Hz, 1 H), 4.71 (d, J=8.0 Hz, 1 H), 4.37 (d, J=7.5 Hz, 1 H), 4.06 (t, J=6.5 Hz, 1 H),4.02(d, J=3.0 Hz, 1H),3.87-3.78 (m, 3 H), 3.72-3.42 (m, 12 H), 3.21 (t, J=8.5 Hz, 1 H); ¹³C NMR (D₂O): δ136.7, 128.9, 129.9, 128.6, 103.0, 101.2, 95.58, 78.8, 77.3, 75.2, 74.9, 74.6, 73.0, 71.7, 70.0, 69.7, 69.4, 69.3, 68.4, 65.0, 61.1, 61.1, 60.3; MS (FAB) 617 (M+Na⁺).

B. Results

1. Cloning, Overexpression and Characterization of Individual Enzymes

As shown in FIG. 1, the biopathway for the synthesis of α-Gal oligosaccharides through UDP-Gal (sugar-nucleotide donor of α1,3GalT) regeneration from UDP (byproduct of the galactosylation reaction) involves five enzymes, including α1,3-galactosyltransferase (α1,3GalT, EC 2.4.1.151), galactoskinase (GalK, EC 2.7.7.6), galactose-1-phosphate uridylyltransferase (GalT, EC 2.7.1.10), glucose-1-phosphate uridylyltransferase (GalU, EC 2.7.1.9), and pyruvate kinase (PykF, EC 2.7.1.40). α1,3GalT catalyzes the synthesis of α-Gal from UDP-Gal and acceptor (lactose or its derivatives). GalK phosphorylates galactose to Gal-1-P with consumption of one molecule of PEP. GalT transfers UDP from UDP-Glc to the galactose in Gal-1-P to produce UDP-Gal. GalU is responsible for the formation of UDP-Glc from UTP and Glc-1-P. The desired galactosylation was catalyzed by an α1,3GalT to transfer the galactose from the donor UDP-Gal to an acceptor and produce the oligosaccharide with the formation of byproduct UDP. PykF recycles UDP to UTP with the consumption of another molecule of PEP.

To obtain active enzymes using recombinant techniques, each of the enzymes involving in the synthetic pathway of α-Gal was individually cloned and overexpressed with a N-terminal His₆-tag introduced by pET15b vector system. The expression of the enzyme was controlled by a T7 lac promoter and induced by 400 μM of IPTG (isopropyl-1-thio-β-D-galactopyranoside). Purification of the enzymes was achieved by passing through a Ni²⁺-NTA affinity column. An SDS-PAGE (sodium dodecyl sulfate polyacrylamide gel electrophoresis) of individual clones indicated that the expression level of each of the four proteins including GalK (A), GalT (B), GalU (C) and PykF (D) was high in pET15b system. The target protein presented more than 80% of the total soluble protein in the host strain. The expression of recombinant α1,3-galactosyltransferase was described in Fang et al. (1998).

The enzyme activity assays were carried out as described in sections (A)(3-6) supra. Repeatedly, 25 U, 100 U, 100 U, and 50 U of GalK, GalT, GalU, and PykF, respectively, could be obtained from 1 l bacteria fermentation.

After the enzyme activities were confirmed by individual assays, the feasibility of the system was determined by two methods. The first one was a quantitative stepwise radioactivity assay. The acceptor for the α1,3GalT in this assay was LacO(CH₂)₇CH₃ (Lac-grease), a lactose derivative containing a hydrophobic part that can bind to the Sep-Pak C₁₈ cartridge. According to the regeneration cycle, radio labeled *Gal was converted to radio-labeled product *Galα1,3 LacO(CH₂)₇CH₃ by stepwise combination of different recombinant enzymes along the pathway (step 1-3 in Table 1). The trisaccharide product was separated from *Gal by passing through Sep-Pak C₁₈ cartridge and eluted with methanol. The radioactivity measured by a scintillation counter presented the amount of the product formed. All of these radioactivity assays were achieved with reasonable high conversions, indicating that each individual recombinant enzyme did function as designed in the regeneration cycle.

The second method to confirm the regeneration cycle was to quantify the formation of α-Gal product by HPLC and characterize the product purified from gel filtration chromatography by NMR and mass spectrometry. Again, stepwise combinations of individual recombinant enzymes with necessary intermediates according to the regeneration pathway were applied. The high yields obtained in these reactions indicated the high efficiency of these enzymes in total regeneration cycle (Table 2).

TABLE 1
Radioactivity assays for the production of α-Gal with purified
recombinant enzymes following the biosynthetic pathway.^{a}

| | | | Product |
|---|---|---|---|
| Steps | Enzymes | Starting Material | (%) |
| 1 | GalK + GalT + α1,3GalT | ATP + Gal + Lac-grease + | 65 |
| | | UDP-Glc | |
| 2 | GalK + GalT + α1,3GalT + | ATP + Gal + Lac-grease + | 50 |
| | GalU | UTP + Glc-1-P (cat.) | |
| 3 | GalK + GalT + α1,3GalT + | ATP + Gal + Lac-grease + | 50 |
| | GalU + PykF | PEP + UDP (cat.) + | |
| | | Glc-1-P (cat.) | |
| ^{a}The acceptor for the α1,3GalT in these assays was LacO(CH₂)₇CH₃ (Lac-grease). It has a hydrophobic part that can bind to the Sep-Pak C₁₈ cartridge. Radio-labeled *Gal was converted to radio-labeled product *Galα1,3LacO(CH₂)₇CH₃ by the combination of recombinant enzymes. The trisaccharide product was separated from *Gal by passing through a Sep-Pak C₁₈ cartridge and eluted with methanol. The radioactivity | | | |
| # measured by a scintillation counter presented the amount of the product formed. | | | |

TABLE 2
Production of Galα1,3LacOH with purified recombinant enzymes
following the biosynthetic pathway.

| | | | Product |
|---|---|---|---|
| | | | (%) by |
| Steps | Enzymes | Starting Material | HPLC |
| 1^{a} | α1,3GalT | Lac + UDP-Gal | 95 |
| 2^{a} | GalT + α1,3GalT | Lac + Gal-1-P + UDP-Glc | 95 |
| 3^{a} | GalK + GalT + α1,3GalT | Lac + ATP + Gal + | 95 |
| | | UDP-Glc | |
| 4^{b} | GalK + GalT + α1,3GalT + | Lac + ATP + Gal + UTP + | 90 |
| | GalU | Glc-1-P (cat.) | |
| 5^{b} | GalK + GalT + α1,3GalT + | Lac + ATP + Gal + PEP + | 90 |
| | GalU + PykF | UDP (cat.) + Glc-1-P (cat.) | |

^{a}The reactions were performed with high concentration (50 mM) of substrates.
^{b}The reactions were performed with substrates at 5 mM. Abbreviations: cat., catalytic amount (0.5 mM).

2. Construction of The Superbug

During whole cell synthesis of α-Gal using E. coli BL21(DE3) strains harboring the individual enzymes, β-galactosidase in the host cell was found to hydrolyzed the acceptor of α1,3GalT (such as lactose) at a high rate. Therefore, an E. coli host strain with a lacZ mutation (lacZ⁻) in which the β-galactosidase was deactivated is preferred for the α-Gal superbug. Thus, another expression vector pLDR20 with a temperature control promoter was used instead. This vector contains an ampicillin resistance gene, a P_{R} promoter and a C₁ repressor gene. The expression of the target gene in pLDR20 was tightly controlled by the temperature. At temperature above 37° C., the C₁ repressor was released from the P_{R} promoter to activate the transcription of the target gene.

The gene of each enzyme involved in the biosynthetic pathway of α-Gal was cloned one by one into pLDR20 vector to form the final plasmid pLDR20-αKTUF. Because there is no ribosomal binding site in the pLDR20 vector bought from ATCC (American Tissue Culture Collection), each of the enzymes was cloned from the corresponding pre-constructed pET 15b-X plasmids with the corresponding N-terminal His6-tag and ribosomal binding sites (FIG. 2). Since GalK and GalT existed in the same gal operon and close to each other, they were cloned together into pET 15b vector and then into the pLDR20 vector. SDS-PAGE indicated that all of the five enzymes in this single plasmid were expressed. The activities of these five enzymes were further confirmed by the synthesis of α-Gal using the enzymes purified from the superbug.

3. Large-Scale Synthesis of α-Gal

After the successful cloning, it was preferable to determine the optimal condition for the synthesis of α-Gal. Therefore, small scale (1 ml) synthesis of α-Gal using the superbug cells was carried out under varied conditions (such as omitting one or more substrates). Either LacOH or LacOBn was used as the acceptor for the α1,3GalT. The α-Gal trisaccharides formation was monitored by thin layer chromatography (TLC) and quantified by high performance liquid chromatography (HPLC). It was found that high yield production of α-Gal was achieved from LacOH (or LacOBn, 25 mM), Gal (50 mM), Glc (50 mM) and catalytic amounts (2 mM) of ATP, Glc-1-P and UDP-Glc. As the case in purified enzyme reaction, high concentration of ATP inhibited the reaction. Furthermore, no PEP was necessary for the whole cell synthesis, although stoichiometric amounts of phosphoenolpyruvate (PEP) and ATP are required for the cell-free in vitro synthesis. This is presumably due to that both PEP and ATP can be generated and recycled through the metabolic pathway of the host E. coli.

Based on the optimized condition obtained from small-scale synthesis, large-scale (5 gram scale) synthesis were successfully carried out. Using the gram scale synthesis of Galα1,3LacOH as an example, α-Gal superbug cells (10 l) were cultured at 30° C. and the expression of the enzymes were induced by increasing the temperature to 40° C. After the expression was continued for 3 hr, the cells were separated from media by centrifugation. Cell pellet (50 g, wet weight) suspended in a Tris-HCl buffer (20 mM, pH 8.5) containing 1% Triton X-100 was added to a reaction mixture of LacOH (4.28 g, 12.5 mmol), Gal (4.50 g, 25 mmol), Glc (4.50 g, 25 mmol), ATP (0.55 g, 1.0 mmol), UDP-Glc (0.61 g, 1.0 mmol), Glc-1-P (0.31 g, 1.0 mmol), MgCl₂ (10 mM), MnCl₂ (10 mM), KCl (100 mM) in HEPES buffer (50 mM, pH 7.5) to a total volume of 500 ml. The reaction was agitated with a magnetic stirrer at room temperature (24° C.) for 36 hr, when thin-layer chromatographic analysis [i-PrOH:NH₄OH:H₂O=7:3:2 (vol/vol/vol)] indicated that reaction was complete. By HPLC analysis, the product Galα1,3LacOH in the reaction mixture was around 10 g/L (80% yield based on acceptor LacOH). After the reaction, the cells were separated from the reaction mixture by centrifugation and washed. Reaction mixture was then passed through an anion exchange column and a cation exchange column and the product was purified by a G-15 sepharose gel filtration column with water as the mobile phase. The trisaccharide-containing fractions were pooled and lyophilized to give Galα1,3LacOH (8.2 g/L).

### Example 2

### Production of Globotriose (Gb3) by Recombinant E. coli Using PykF

This example describes the construction of a metabolic pathway-engineered E. coli strain, referred to as a superbug, harboring all the enzymes in the biosynthetic pathway of globotriose. PykF was incorporated using PEP as energetic source. Also, described is the use of the superbug to produce oligosaccharides with a terminal Galα,4Gal sequence.

A. Materials and Methods Used in this Example

1. Bacterial Strains and Plasmids

Same as in the Example 1.

2. Cloning, Overexpression and Purification of Individual Enzymes

DNA manipulations were performed as described in Example 1. For construction and overexpression of a recombinant α1,4GaIT from N. meningitidis, IgtC gene (primers 5′-CGGAATTCATATGGACATCGTATTTGCG-3′(SEQ ID NO: 16) and 5′-GCCGGATCCTCATCAGTGCGGGACGGCAAGTTTGCC-3′) (SEQ ID NO: 17)) was cloned from N. meningitidis MC58 with a deletion of the codon sequence encoding for the 25 amino acids at the C-terminal of the full length LgtC protein. The PCR amplified product was purified by QIAquick PCR Purification Kit and QIAEX II Gel Extraction Kit (Qiagen, Santa Clarita, Calif.), digested with Nde I and BamH I restriction enzymes and inserted into pET15b vector. The resulting plasmid pET 15b-lgtC-25aa was transformed into E. coli cloning host strain DH5a and then expression host strain BL21(DE3), respectively. Selected clones were characterized by restriction mapping. The expression and purification of LgtC were as described in the cloning, overexpression and purification of individual enzymes in Example 1.

3. Enzyme Activity Assay for LgtC

Enzyme assays for LgtC were performed at 37° C. for 15 min in a final volume of 100 μl containing Tris-HCl (10 mM, pH 7.0), MnCl₂ (10 mM), DTT (5 mM), bovine serum albumin (0.1%), UDP-D-[6-³H]galactose (0.3 mM) (final specific activity of 1000 cpm/nmol), LgtC (20 μl), and lactose (50 mM). Lactose was omitted for blank. The reaction was stopped by adding 100 μl of ice-cold EDTA (0.1 M). Dowex 1×8-200 chloride anion exchange resin was then added in a water suspension (0.8 ml, 1:1 (v/v)). After centrifugation, supernatant (0.5 ml) was collected in a 20-mi plastic vial, and SciintiVerse BD (5 ml) was added. The vial was vortexed thoroughly before the radioactivity of the mixture was counted in a liquid scintillation counter (Beckmann LS-3801 counter). One unit of fusion enzyme activity is defined as the amount of enzyme that catalyzes the transfer of 1 μmol of galactose from UDP-Gal to lactose per min at 37° C.

4. Construction of the Pathway-Engineered Organism (Superbug)

The plasmid for the CKTUF superbug was constructed in a similar manner as described in experiment 1 into the pLDR20 plasmid vector. Primers α1,3GaIT-N (5′-GGATCCATATGACTAGTGATATCAATAATTTTGTTTAACTTTAAGAAGG-3′ (SEQ ID NO: 12)) and IgtC-C (5′-TCCCCGCGGTCATCAGTGCGGGACGGCAAGTTTGCC -3′ (SEQ ID NO: 18)) were used to amplify the IgtC gene with ribosomal binding site and codons for HiS₆-tag from pre-constructed plasmid pET15b-lgtC-25aa. The PCR product was digested and inserted into EcoR V and Sac II two restriction sites of the plasmid pLDR20-U (constructed in Example 1) to form plasmid pLDR20-CU. Then, pykF gene and galK+galT genes were inserted as described in Example 1 to form plasmid pLDR20-CKTUF. This final plasmid harboring five genes was transformed into DH5α and NM522 competent cells.

5. Gram-Scale Synthesis of Galα1,4GalOR with Superbug

The expression and preparation of cell suspension were as described in Example 1. Gram-scale synthesis was performed with a variety of galactose or lactose derivatives as the acceptor for the LgtC. In a 250 ml flask was added acceptor (2.92 mmol), Gal (1.05 g, 5.84 mmol), Glc (1.05 g, 5.84 mmol), ATP (129 mg, 0.234 mmol), UDP-Glc (143 mg, 0.234 mmol), Glc-1-P (72 mg, 0.234 mmol), and 12 ml of each of the following stock solutions: HEPES buffer (0.5 M, pH 7.4), MnCl₂ (0.1 M), MgCl₂ (0.1 M), and KCI (1 M). Then superbug cells [12 g in 72 ml Tris-HCl buffer (20 mM, pH 8.5) containing 1% Triton X-100, obtained from 21 bacterial culture] was added to bring the total reaction mixture volume to 120 ml. The reaction was agitated with a magnetic stirrer at room temperature (24° C.) for 36 h. The reaction was monitored by TLC [i-PrOH:H2O:NH₄OH=7:3:2 (vol/vol/vol)] and high performance liquid chromatography (HPLC). After 36 hr, the reaction was stopped by putting the flask in boiling water for 10 min. The pellet was removed by centrifugation at 5,000×g for 20 min and washed twice with 50 ml deionized water. The combined supernatants were passed through an anion exchange column and a cation exchange column. The concentrated eluent was loaded to a Sephadex G-15 gel filtration column (120 cm×4 cm) with water as the mobile phase. The desired fractions were pooled and lyophilized to give the derivatives of globotriose.

B. Results

1. Cloning, Overexpression and Characterization of LgtC

LgtC catalyzes the transfer of galactose from the donor UDP-Gal to an acceptor and produce the oligosaccharide with the formation of byproduct UDP. Repeatly, LgtC was overexpressed in a high yield as an active soluble form in the cell lysate. About 300 U purified enzyme can be obtained from 1 liter E. coli culture. Globotriose (Gb₃, the sugar sequence of Galα1,4Galβ 1,4Glc) is a trisaccharide portion of globotriaosylceramide, the receptor of E. coli-derived verotoxin (VT). VT binding to the Gb₃ is believed to be a crucial step in the development of hemorrhagic colitis, and hemolytic uremic syndrome commonly known as ‘Hamburger disease’ (Lingwood, Nephron. 1994, 66, 21-28. Lingwood, C. A. Biochim. Biophys. Acta 1999, 1455, 375. Peter, M. G.; Lingwood, C. A. Biochim. Biophys. Acta 2000, 1501, 116. Barnett, F. D.; Abul-Milh, M.; Huesca, M.; Lingwood, C. A. Infect. Immun. 2000, 68, 3108. Lingwood, C. A. Biosci. Rep. 1999, 19, 345.). Gb₃ plays a direct role in Shiga toxin entry into the cell though the interaction of B-subunit of Shiga toxins and Gb₃. (Lindberg et al., J. Biol. Chem. 1987, 262, 1779-1785.) Synthetic Gb₃ derivatives could be effective inhibitors of these interactions and have important pharmaceutical potential. Gb₃ was also identified as P^{k} blood group antigen (Marcus et al., Semin. Hematol. 1981, 18, 63-71.) and was found in the LOS (lipooligosaccharides) of the pathogens Neisseria meningitidis immunotype L1 and N. gonorhoeae (Scholten et al., Med. Microbiol. 1994, 41, 236-243. Jennings et al., Mol. Microbiol. 1995, 18, 729-740.). Large amount of Gb₃ is essential for the experimental and clinical research on preventing pathogen invasion.

2. Gram-Scale Synthesis of Galα1,4GalOR

The biopathway for the synthesis of Gb₃ through the regeneration UDP-Gal includes five enzymes, including LgtC and four enzymes involved in the regeneration of UDP-Gal such as galactoskinase (GalK), galactose-1-phosphate uridylyltransferase (GalT), glucose-1-phosphate uridylyltransferase (GalU), and pyruvate kinase (PykF). Based on the small scale (1 ml) synthesis of Gb₃, the optimized condition for the CKTUF superbug catalyzed reaction was found as following: Acceptor (25 mM), Gal (50 mM), Glc (50 mM), MnCl₂ (10 mM), MgCl₂ (10 mM), KCl (100 mM) and catalytic amounts (2 mM) of ATP, Glc-1-P and UDP-Glc with catalytic amount (5 mM) of PEP in 50 mM of HEPES buffer, pH 7.5. Since both PEP and ATP can be generated and recycled through the metabolic pathway of the host E. coli, only catalytic amount of PEP and ATP are needed for the high-yield whole cell synthesis, although stoichiometric amounts of phosphoenolpyruvate (PEP) and ATP are required for the cell-free in vitro synthesis. The scope of superbug technology was explored in gram-scale synthesis of Gb₃ derivatives (Table 3). Cell pellet (10 g, wet weight) suspended in a Tris-HCI buffer (20 mM, pH 8.5) containing 1% Triton X-100 was added to a reaction mixture of acceptor (2.92 mmol), Gal (1.05 g, 5.84 mmol), Glc (1.05 g, 5.84 mmol), ATP (129 mg, 0.234 mmol), UDP-Glc (143 mg, 0.234 mmol), Glc-1-P (72 mg, 0.234 mmol), MgCl₂ (10 mM), MnCl₂ (10 mM), KCl (100 mM) in HEPES buffer (50 mM, pH 7.5) to a total volume of 120 ml. The reaction was agitated with a magnetic stirrer at room temperature (24° C.) for 36 hr, when thin-layer chromatographic analysis [i-PrOH:NH₄OH:H₂O=7:3:2 (vol/vol/vol)] indicated that reaction was complete. Then, the cells were separated from the reaction mixture by centrifugation and washed. Reaction mixture was then passed through an anion exchange column and a cation exchange column and the product was purified by a Sephadex G-5 gel filtration column with water as the mobile phase. The product-containing fractions were pooled and lyophilized. Table 3 indicates that the superbug can accept a variety of oligosaccharides as the substrate for the synthesis of globotriose derivatives. Lactose derivatives are good acceptors; galactose derivatives are worse acceptors.

TABLE 3
Gram-Scale Production of Galal,4GalOR with Superbug CKTUF

| Entry | Acceptor | Yields (%) | Entry | Acceptor | Yields (%) |
|---|---|---|---|---|---|
| 1 | 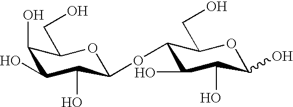 | 75 | 5 | 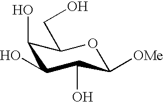 | 10 |
| 2 | 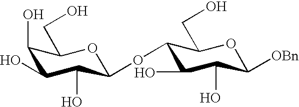 | 85 | 6 | 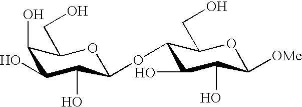 | 50 |
| 3 | 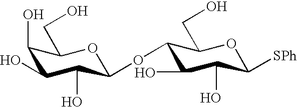 | 50 | 7 | 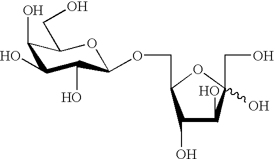 | 45 |
| 4 | 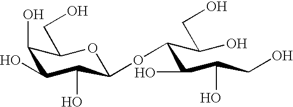 | 20 | 8 | 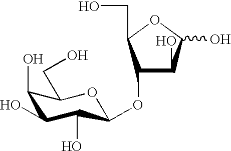 | 60 |

### Example 3

### Production of α-Gal Epitopes by Recombinant E. coli Using Sucrose Synthase

This example describes the construction of a metabolic pathway-engineered E. coli strain, referred to as a superbug, harboring all the enzymes in the biosynthetic pathway of α-Gal. Sucrose synthase was incorporated using sucrose for the regeneration of UDP-Gal. Also, described is the use of the superbug to produce oligosaccharides with a terminal Galα1,3Gal sequence.

A. Materials and Methods Used in this Example

1. Bacterial Strains and Plasmids

Same as in the Example 1.

2. Cloning, Overexpression and Purification of Sucrose Synthase (SS) and UDP-Gal 4-Epimerase (GalE)

DNA manipulations were performed as described in Example 1. For construction and overexpression of a recombinant sucrose synthase (SS), susA gene (primers 5′- CCGCTCGAGATGTCAGAATTGATGCAAGCG-3′ (SEQ ID NO: 19) AND 5′-CGCGGATCCTTACCGATATTTATGCTG-3′) (SEQ ID NO: 20)) was cloned from cyanobacteria Nanabaena sp. Strain PCC 7119 (ATCC no. 29151). The PCR amplified product was purified, digested with Xho I and BamH I restriction enzymes, and inserted into pET 15b vector. The resulting plasmid pET 15 b-susA was transformed into E. coli cloning host strain DH5α and then expression host strain BL21 (DE3), respectively. Selected clones were characterized by restriction mapping. The expression and purification of SS were as described in the cloning, overexpression and purification of individual enzymes in Example 1.

The construction, expression, and characterization of a recombinant GalE from E. coli were as described in Chen et al. (Biotechnology Letters 1999, 21, 1131-1135).

3. Enzyme Activity Assay for SS

Enzyme assays for SS were performed at 37° C. for 15 min in a final volume of 100 μl containing MES (50 mM, pH 6.0), MgCl₂ (10 mM), DTT (5 mM), UDP-D-[6-³H]glucose (0.3 mM) (final specific activity of 1000 cpm/nmol), SS (20 μl), and fructose (50 mM). Fructose was omitted for blank. The reaction was stopped by adding 100 μl of ice-cold EDTA (0.1 M). Dowex 1×8-200 chloride anion exchange resin was then added in a water suspension (0.8 ml, 1:1 (v/v)). After centrifugation, supernatant (0.5 ml) was collected in a 20-ml plastic vial, and ScintiVerse BD (5 ml) was added. The vial was vortexed thoroughly before the radioactivity of the mixture was counted in a liquid scintillation counter (Beckmann LS-3801 counter). One unit of fusion enzyme activity is defined as the amount of enzyme that catalyzes the transfer of 1 μmol of glucose from UDP-Glc to fructose per min at 37° C.

4. Construction of the Pathway-Engineered Organism (Superbug)

The plasmid for the αES superbug was constructed based on the plasmid constructed in Example 1. Primers GalU-N (5′-CCGGATATCCCGCGGGTCGACAATAATTTTGTTTAACTTTAAGAAGG-3′ (SEQ ID NO: 10)) and GalE-C (5′-CGCGGATCCGCATGCTTAATCGGGATATCCCTG-3′ (SEQ ID NO: 21) (introducing Sph I and BamH I restriction sites) were used to amplify the galE gene with ribosomal binding site and codons for His₆-tag from pre-constructed plasmid pET15b-galE. The PCR product was digested with Sal I and BamH I restriction enzymes and inserted into plasmid pLDR20-αUF constructed in Example 1 to form plasmid pLDR20-αE. Primers SusA-N (5′-GATCGCATGCAATAATTTTGTTTAACTTTAAGAAGG-3′ (SEQ ID NO: 22)) and SusA-C (5′-CGCGGATCCTTACCGATATTTATGCTG-3′ (SEQ ID NO: 23)) were used to amplify the susA gene with ribosomal binding site and codons for His₆-tag from pre-constructed plasmid pET15b-susA. The PCR product was digested and inserted into Sph I and BamH I two restriction sites of the plasmid pLDR20-αE to form plasmid pLDR20-αES. This final plasmid harboring three genes was transformed into DH5α and NM522 competent cells.

5. Gram-Scale Synthesis of Galα1,4GalOR with Superbug

The expression and preparation of cell suspension were as described in Example 1. Gram-scale synthesis was performed with lactose as the acceptor for the α1,3GalT. In a 150 ml flask was added LacOH (1.00 g, 2.92 mmol), sucrose (1.40 g, 4.09 mmol), UDP-Glc (358 mg, 0.585 mmol) and 6 ml of each of the following stock solutions: MES buffer (0.5 M, pH 6.0), and MgCl₂ (0.1 M). Then superbug cells [8 g in 30 ml Tris-HCI buffer (20 mM, pH 8.5) containing 1% Triton X-100, obtained from 1.5 l bacterial culture] was added, and H₂0 was added to bring the total reaction mixture volume to 60 ml. The reaction was agitated with a magnetic stirrer at room temperature (24° C.) for 36 hr. The reaction was monitored by TLC [i-PrOH:H₂O:NH₄OH=7:3:2 (vol/vol/vol)] and high performance liquid chromatography (HPLC). After 36 hr, the reaction was stopped by putting the flask in boiling water for 10 min. The pellet was removed by centrifugation at 5,000×g for 20 min and washed twice with 50 ml deionized water. The combinaed supernatants were passed through an anion exchange column and a cation exchange column consequently. The concentrated eluent was loaded to a Sephadex G-15 gel filtration column (120 cm×4 cm) with water as the mobile phase. The desired fractions were pooled and lyophilized to give the derivatives of globotriose.

B. Results

1. Cloning Overexpression and Characterization of SusA

Repeatly, about 10 U/l SusA (sucrose synthesis direction) was obtained from the cell lysate.

2. Gram-Scale Synthesis of Galα1,3LacOH

The biopathway for the synthesis of α-Gal through the regeneration UDP-Gal using sucrose as energetic (FIG. 15) includes three enzymes, including al,3 GaIT and two enzymes involved in the regeneration of UDP-Gal such as UDP-galactose 4-epimerase (GalE) and sucrose synthase (SS). Based on the small scale (1 ml) synthesis, the optimized condition for the αES superbug catalyzed reaction was found as following: LacOH (50 mM), Suc (70 mM), MgCl₂ (10 mM), and catalytic amounts (5 mM) of UDP-Glc in 50 mM of MES buffer, pH 6.0. This cycle is one of the simplest pathways for the synthesis of a-Gal through UDP-Gal regeneration.

Gram-scale synthesis of a-Gal was carried out as described above. Unlike the low yield obtained using the combination of purified GalE, αl,3GalT and SS due to the instability of the SS, high yields (75-85%) were obtained using the superbug catalyzed synthesis. Although DTT is required for the purified SS catalyzed sucrose cleavage reaction in vitro, no DTT is necessary for the superbug. These are additional advantages of the superbug strategy.

### Example 4

### Production of Galα1,4GalOR by Recombinant E. coli Using Sucrose Synthase

This example describes the construction of a metabolic pathway-engineered E. coli strain, referred to as a superbug, harboring all the enzymes in the biosynthetic pathway of globotriose. Sucrose synthase was incorporated using sucrose for the regeneration of UDP-Gal. Also, described is the use of the superbug to produce oligosaccharides with a terminal Galα1,4Gal sequence.

A. Materials and Methods Used in this Example

1. Bacterial Strains and Plasmids

Same as in Example 1.

2. Cloning Overexpression and Purification of Sucrose Synthase (SS) and UDP-Gal 4-Epimerase (GalE)

Same as in Example 3.

3. Construction of the Pathway-Engineered Organism (Superbug)

The plasmid for the CES superbug was constructed based on the plasmids constructed in Examples 2 and 3. The PCR product of gale, as in Example 3, was digested with Sal I and BamH I restriction enzymes and inserted into plasmid pLDR20-CUF constructed in Example 2 to form plasmid pLDR20-CE. The PCR product of susA, as in Example 3, was digested and inserted into Sph I and BamH I two restriction sites of the plasmid pLDR20-CE to form plasmid pLDR20-CES. This final plasmid harboring three genes was transformed into DH5α and NM522 competent cells.

4. Gram-Scale Synthesis of Galα1,4GalOR with Superbug

The expression and preparation of cell suspension were as described in Example 1. Gram-scale synthesis was performed with lactose as the acceptor for the LgtC. In a 150 ml flask was added LacOH (1.00 g, 2.92 mmol), sucrose (1.40 g, 4.09 mmol), UDP-Glc (358 mg, 0.585 mmol) and 6 ml of each of the following stock solutions: MES buffer (0.5 M, pH 6.0), and MgCl₂ (0.1 M). Then superbug cells [8 g in 30 ml Tris-HCI buffer (20 mM, pH 8.5) containing 1% Triton X-100, obtained from 1.5 l bacterial culture] was added, and H₂O was added to bring the total reaction mixture volume to 60 ml. The reaction was agitated with a magnetic stirrer at room temperature (24° C.) for 36 hr. The reaction was monitored by TLC [i-PrOH:H₂O:NH₄OH=7:3:2 (vol/vol/vol)] and high performance liquid chromatography (HPLC). After 36 hr, the reaction was stopped by putting the flask in boiling water for 10 min. The pellet was removed by centrifugation at 5,000×g for 20 min and washed twice with 50 ml deionized water. The combined supernatants were passed through an anion exchange column and a cation exchange column consequently. The concentrated eluent was loaded to a Sephadex G-15 gel filtration column (120 cm×4 cm) with water as the mobile phase. The desired fractions were pooled and lyophilized to give the derivatives of globotriose.

B. Results

1. Gram-Scale Synthesis of Galα1,4LacOH

The biopathway for the synthesis of globotriose through the regeneration UDP-Gal using sucrose as energetic includes three enzymes, including LgtC and two enzymes involved in the regeneration of UDP-Gal such as UDP-galactose 4-epimerase (GalE) and sucrose synthase (SS). Based on the small scale (1 ml) synthesis, the optimized conditions for the CES superbug catalyzed reaction was similar to those for the αES superbug as follows: LacOH (50 mM), Suc (70 mM), MgCl₂ (10 mM), and catalytic amounts (5 mM) of UDP-Glc in 50 mM of MES buffer, pH 6.0. Gram-scale synthesis of globotriose was carried out as described above. Quantitative yields (85-95%) were obtained using the CES superbug. No DTT is required.

The references cited in this disclosure, except in which they may contradict any statements or definitions made herein, are incorporated by reference in their entirety.

The following table lists abbreviations used herein (Table 4).

TABLE 4
Abbreviations

| Abbreviation | Definition |
|---|---|
| AcK | Acetate kinase |
| ADP | adenosine 5′-diphosphate |
| Alg1 | GDP-Man: Dol-PP-GlcNAc β-mannosyltransferase |
| Alg2 | α1,3-mannosyltransferase |
| ATP | adenosine 5′-triphosphate |
| Cmk | CMP kinase |
| CMP | Cytosine 5′-monophosphate |
| CMP-NeuNAc | Cytosine 5′-monophospho-N-acetylneuraminic acid |
| cpsG(manS) | encodes PMM |
| cpsB(manC) | encodes GMP |
| CTP | Cytosine 5′-triphosphate |
| dATP | deoxyadenosine 5′-triphosphate |
| dCMP | deoxycytosine 5′-monophosphate |
| Eagle's MEM | Eagle's minimum essential medium |
| EDTA | Ethylenediaminetetraacetic acid |
| FucOR | Fucose terminated glycoconjugate |
| FucT | fucosyltransferase |
| Glk | glucose kinase |
| Gal | galactose |
| Gal-1-P | galactose-1-phosphate |
| GalE | UDP-Gal 4-Epimerase, UDP-Glc 4-Epimerase |
| GalK | galactokinase |
| GalNAc | N-acetylgalactosamine |
| GalNAc-1-P | N-acetylgalactosamine-1-phosphate |
| GalT | galactose-1-phosphate uridylyltransferase |
| GalU | glucose-1-phosphate uridylyltransferase |
| GDP-Fuc | Guanosine 5′-diphosphofucose |
| GDP-Man | Guanosine 5′-diphosphomannose |
| GFS | GDP-L-fucose synthetase |
| Glc-1-P | glucose-1-phosphate |
| GlcA | Glucuronic acid |
| GlcNAc | N-acetylglucosamine |
| GlcNAcOR | Glycoconjugate terminated with N- |
| | acetylglucosamine |
| GlcOR | Glycoconjugate terminated with glucose |
| GMD | GDP-D-mannose 4,6-dehydratase |
| GMER | GDP-4-keto-6-deoxy-D-mannose |
| | epimerase/reductase |
| GMP | GDP-mannose pyrophosphorylase |
| GST | Glutathione S-transferase |
| GTP | Guanosine 5′-triphosphate |
| HAS | hyaluronan synthases |
| HEPES | N-(2-Hydroxyethyl)piperazine-N′-(2-ethanesulfonic |
| | acid) |
| HPLC | high performance liquid chromatography |
| IAA | indoleacrylic acid |
| LgtA | β1,3GlcNAc transferase |
| IPTG | isopropyl-β-D-thiogalactopyranoside |
| IRES | internal ribosome entry site |
| ITP | Inositol-5′-triphosphate |
| Lac | lactose |
| Lac-grease | LacO(CH₂)₇CH₃ |
| LacNAc | N-acetylactosamine |
| lacZ | β-galactosidase |
| LPS | lipopolysaccharide |
| LPS O-antigen | lipopolysaccharide O antigen |
| manB | Phosphomannomutase gene |
| manC | mannose-1-phosphate guanyltransferase gene, GDP- |
| | mannose pyrophosphorylase gene |
| ManNAc | N-acetylmannosamine |
| ManOR | Glycoconjugate terminated with mannose |
| NAD | Nicotinamide adenine dinucleotide |
| NADH | Nicotinamide adenine dinucleotide (reduced form) |
| NanA | N-acetylneuraminate lyase, sialic acid aldolase |
| nanA | sialic acid aldolase gene |
| NeuA | CMP-NeuNAC synthetase |
| neuA | CMP-NeuNAc synthetase |
| NeuAc | N-acetylneuraminic acid |
| NeuNAc | N-acetylneuraminic acid |
| nickel-NTA | nickel-nitrilotriacetic acid |
| NMR | nuclear magnetic resonance |
| OD | optical density |
| PEP | phospho(enol)pyruvate |
| PgM | phosphoglucomutase |
| PMI | phosphomannose isomerase |
| PMM | phosphomannomutase |
| PoxB | Pyruvate oxidase |
| ppa | Pyrophosphatase gene |
| PPase | pyrophosphatase |
| PPi | pyrophosphate |
| PpK | polyphosphate kinase |
| PTS | PEP-dependent transporter system |
| PykA | pyruvate kinase |
| PykF | pyruvate kinase |
| rbs | ribosomal binding site |
| RBS | ribosome binding sequence |
| rfbK | encodes PMM |
| rfbM | encodes GDP-mannose pyrophosphorylase |
| SDS-PAGE | sodium dodecyl sulfate polyacrylamide gel |
| | electrophoresis |
| SiaT | α2,3 (or α2,6)-sialyltransferase |
| spHas | Hyaluronan synthase from Streptococcus pyogenes |
| SS | sucrose synthase |
| susA | Sucorose synthetase gene |
| UDP | uridine 5′-diphosphate |
| UDP-Gal | uridine 5′-diphosphogalactose |
| UDP-GlcA | uridine 5′-diphosphoglucuronic acid |
| UDP-GalNAc | uridine 5′-diphospho-N-acetylgalactosamine |
| UDPGDH | UDP-Glc 6-dehydrogenase |
| UDP-GlcNAc | uridine 5′-diphospho-N-acetylglucosamine |
| UDP-Glc | uridine 5′-diphosphoglucose |
| UGT | UDP-glucuronosyltransferase |
| UTP | uridine 5′-triphosphate |
| α2,6SiaT | SiaT 0160 |
| α-Gal | α-galactose epitopes |

1. A vector comprising:
(a). two or more genes encoding sugar-nucleotide regenerating enzymes selected from the group consisting of GalK, GalT, GalU, PykF, Ndk, PpK, AcK, PoxB, Ppa, PgM, NagE, Agm1, glmU, a GalNAc kinase, a pyrophosphorylase, Ugd, NanA, Cmk, NeuA, Alg2, Alg1, SusA, ManB, ManC, a phosphomannomutase, GalE, GMP, GMD, and GFS; and
(b). one or more genes encoding glycosyltransferase(s),
wherein said genes are operably linked to a promoter.

2. The vector of claim 1 comprising genes encoding three or more enzymes for regenerating a sugar-nucleotide.

3. The vector of claim 1 comprising genes encoding two or more glycosyltransferases.

4. The vector of claim 1 comprising genes encoding three or more glycosyltransferases.

5. The vector of claim 1 comprising genes encoding GalK, GalT, and GalU.

6. The vector of claim 5 further comprising a gene encoding Ndk.

7. The vector of claim 5 further comprising a gene encoding Ppk.

8. The vector of claim 5 further comprising a gene encoding PykF.

9. The vector of claim 5 further comprising genes encoding PoxB, Ndk, and Ppa.

10. The vector of claim 1 comprising a gene encoding SusA.

11. The vector of claim 10 further comprising a gene encoding GalE.

12. The vector of claim 10 further comprising a gene encoding GluT.

13. The vector of claim 10 further comprising genes encoding Ugd and UGT2B7.

14. The vector of claim 1, wherein the one or more glycosyltransferase(s) is selected from the group consisting of a galactosyltransferase, a glucosyltransferase, an N-acetylglucosaminyl transferase, an N-acetylgalactosaminyl transferase, a glucuronyltransferase, a sialyltransferase, a mannosyltransferase, and a fucosyltransferase.

15. The vector of claim 14, wherein the galactosyltransferase is selected from the group consisting of LgtB and LgtC.

16. The vector of claim 14, wherein the glucosyltransferase is selected from the group consisting of LgtF, Alg5, and DUGT.

17. The vector of claim 14, wherein the N-acetylglucosaminyl transferase is LgtA.

18. The vector of claim 14, wherein the N-acetylgalactosaminyl transferase is UDP-GalNAc:2′-fucosylgalactoside-α-3-N-acetylgalactosaminyl transferase.

19. The vector of claim 14, wherein the glucuronyltransferase is UGT2B7.

20. The vector of claim 14, wherein the sialyltransferase is SiaT 0160.

21. The vector of claim 14, wherein the mannosyltransferase is selected from the group consisting of Alg1 and Alg2.

22. The vector of claim 14, wherein the fucosyltransferase is selected from the group consisting of α1,3-FucT, α1,2-FucT, and α1,3/4-FucT.

23. The vector of claim 1 wherein the promoter is an inducible promoter.

24. The vector of claim 23, wherein the inducible promoter is λ P_{R} promoter.

25. The vector of claim 24 further comprising a λ C₁ repressor gene.

26. The vector of claim 1, wherein at least one gene is operably linked to a ribosomal binding site sequence.

27. The vector of claim 26, wherein each gene encoding a sugar-nucleotide regenerating enzyme or a glycosyltransferase is operably linked to a ribosomal binding site sequence.

28. The vector of claim 1, wherein at least one gene is operably linked to an IRES.

29. The vector of claim 1, wherein at least one gene is operably linked to a tag sequence.

30. The vector of claim 29, wherein each gene encoding a sugar-nucleotide regenerating enzyme or a glycosyltransferase is operably linked to a tag sequence.

31. The vector of claim 29, wherein the tag sequence encodes polyhistidine.

32. The vector of claim 1, wherein the vector encodes an epimerase.

33. The vector of claim 1, wherein the vector encodes a fusion protein.

34. The vector of claim 33, wherein the fusion protein comprises an epimerase and a glycosyltransferase.

35. The vector of claim 34, wherein the epimerase is UDP-Gal-4-epimerase.

36. The vector of claim 35, wherein the glycosyltranferase is α-1,3-galactosyltransferase.

37. The vector of claims 1, wherein the vector is selected from the group consisting of plasmids, phage, phagemids, viruses, and artificial chromosomes.

38. The vector of claim 37, wherein the vector is a plasmid.

39. A cell comprising heterologous genes encoding one or more sugar-nucleotide regenerating enzyme and one or more glycosyltransferase.

40. The cell of claim 39, wherein the cell is a prokaryotic cell.

41. The cell of claim 40, wherein the prokaryotic cell is a bacterium.

42. The cell of claim 41, wherein the bacterium is E. coli.

43. The cell of claim 42, wherein the E. coli is LacZ⁻.

44. The cell of claim 39, wherein the cell is a eukaryotic cell.

45. The cell of claim 44, wherein the eukaryotic cell is a yeast.

46. The cell of claim 39, wherein at least one of the heterologous genes is integrated into the genome of the cell.

47. The cell of claim 39, wherein the heterologous genes are encoded within one or more plasmids.

48. The cell of claim 47, wherein the heterologous genes are encoded within one plasmid.

49. A method of producing a glycoconjugate comprising the step of contacting a cell comprising heterologous genes encoding:
(i). one or more encoding sugar-nucleotide regenerating enzymes selected from the group consisting of GalK, GalT, GalU, PykF, Ndk, PpK, AcK, PoxB, Ppa, PgM, NagE, Agm1, glmU, a GalNAc kinase, a pyrophosphorylase, Ugd, NanA, Cmk, NeuA, Alg2, Alg1, SusA, ManB, ManC, a phosphomannomutase, GalE, GMP, GMD, and GFS; and
(ii). one or more glycosyltransferase,
with a bioenergetic.

50. A kit comprising the plasmid of claim 1.

51. A non-human cell comprising the plasmid of claim 1.
